(19) United States
(12) Patent Application Publication
(10) Pub. No.: US 2014/0194517 A1
(43) Pub. Date: Jul. 10, 2014

(54) Treatment of Graft-Versus-Host Disease Disorders using RAR Antagonists

(71) Applicants: Io Therapeutics, Inc., Laguna Hills, CA (US); The Regents of the University of Minnesota, Minneapolis, MN (US)

(72) Inventors: Bruce Blazer, Golden Valley, MN (US); Kazutoshi Aoyama, St. Anthony, MN (US); Roshantha Chandraratna, Laguna Hills, CA (US)

(73) Assignees: Io Therapeutics, Inc., Laguna Hills, CA (US); The Regents of the University of Minnesota, Minneapolis, MN (US)

(21) Appl. No.: 14/150,574

(22) Filed: Jan. 8, 2014

Related U.S. Application Data
(60) Provisional application No. 61/750,039, filed on Jan. 8, 2013.

(51) Int. Cl. A61K 31/196; A61K 31/195

(52) U.S. Cl. CPC A61K 31/196; A61K 31/195
USPC 514/563; 514/567

(57) ABSTRACT

The present specification provides RAR antagonist compounds, compositions comprising such RAR antagonists, and methods using such compounds and compositions to treat an autoimmune disorder, inflammation associated with an autoimmune disorder and/or a transplant rejection as well as use of such RAR antagonists to manufacture a medicament and use of such compounds and compositions to treat an autoimmune disorder, inflammation associated with an autoimmune disorder and/or a transplant rejection.

### CROSS-REFERENCE TO RELATED APPLICATIONS

This application claims the benefit under to U.S. Provisional Patent Application 61/750,039 filed Jan. 8, 2013, the entire contents of which is incorporated by reference herein.

### GOVERNMENT SUPPORT

This invention was made with government support under R01-A134495, R01-HL56067, P01-A1056299, and R01-CA062275 awarded by National Institutes of Health (NIH). The government has certain rights in the invention.

### FIELD

Disclosed herein are methods of treating graft-versus-host disease with RAR antagonists.

### BACKGROUND

Graft-versus-host disease (GVHD) is a substantial cause of morbidity and mortality after allogeneic bone marrow transplantation (BMT). Conditioning regimen injury creates a pro-inflammatory environment that recruits donor T effectors (Teff), which result in gut injury leading to GVHD lethality. Neutralizing pro-inflammatory cytokines reduces but does not eliminate gut injury, indicating the importance of other pathways.

Attempts to treat GVHD disorders have met with limited success. This is due, in part, to the fact that the etiology of GVHD disorders is a complex response based in part on a combination of factors, including, without limitation, genetic make-up of individual, gender or hormonal status, bacterial or viral infection, metal or chemical toxin exposure, vaccinations or immunizations, stress, trauma, smoking and/or nutritional deficiencies. Therefore, compounds, compositions, and methods that can reduce a symptom associated with a GVHD disorder, inflammation associated with a GVHD disorder, and/or a transplant rejection would be highly desirable.

Naïve CD4⁺ T cells play a central role in immune protection. They do so through their capacity to help B cells make antibodies, to induce macrophages to develop enhanced microbicidal activity, to recruit neutrophils, eosinophils, and basophils to sites of infection and inflammation, and, through their production of cytokines and chemokines, to orchestrate the full panoply of immune responses. Naïve CD4⁺ T cells are multipotential precursors that differentiate into various T cell subsets, such as, e.g., T helper (Th) cells (also called T effector cells) and T regulatory (Treg) cells. T helper cells are characterized by their distinct functions and include Th1, Th2, and Th17. Th1 cells aid in the clearance of intracellular bacteria and viruses, secrete IFN-γ in response to the cytokine interleukin-12 (IL-12), and require the transcription factors T-box21 (T-bet) and signal transducer and activator of transcription 1 (Stat1) and (Stat4). Th2 cells help control extracellular pathogens, secrete the cytokines IL-4, IL-5 and IL-13, and require transcription factors GATA-binding protein 3 (GATA-3) and Stat6. Th17 cells provide protection against fungi and various other extracellular bacteria, secrete the pro-inflammatory cytokine IL-17A, and express the transcription factor retinoic acid orphan receptor gamma (RORγt). Treg cells play a critical role in maintaining self-tolerance as well as in regulating immune responses and express the transcription factor forkhead box P3 (FoxP3). Tregs normally develop in the thymus, but can also differentiate from naïve CD4⁺ cells stimulated with TGF-β and IL-2. Development and differentiation of Treg cells, as well as expression of FoxP3, require the transcription factor Stat5.

Although several cytokines participate in Th17 cell differentiation, IL-6 and TGF-β are key factors for the generation of Th17 cells from naïve T CD4⁺ cells. On the other hand, IL-6 inhibits TGF-β-induced Treg cells which suppress adaptive T cell responses and prevent autoimmunity, and are thus important in the maintenance of immune homeostasis. The two T cell subsets play prominent roles in immune functions: Th17 plays a key role in the pathogenesis of autoimmune diseases and protection against bacterial infections, while Treg functions to restrain excessive helper T cell responses. Essentially immunosuppressive Tregs cells and pro-inflammatory Th17 cells functionally antagonize each other.

As such, a fine balance between Th17 and Treg cells may be crucial for the stability of immune homeostasis. Once the equilibrium is broken, the destabilization may lead to chronic inflammation and autoimmunity. For example, dysregulation or overproduction of IL-6 leads to autoimmune diseases such as multiple sclerosis (MS) and rheumatoid arthritis, in which Th17 cells are considered to be the primary cause of pathology. Clinical evidence indicates that both defects in Treg function or reduced numbers, as well as Th17 activity are important in several autoimmune diseases, including seronegative arthritis in adults, and childhood arthritis (juvenile idiopathic arthritis). Therefore, an effective approach in the treatment of various autoimmune and inflammatory diseases will be to normalize the balance between Treg and Th17 cell development.

Retinoic acid (RA) appears to exert a powerful impact on the differentiation of T cells and other leukocytes as well as intestinal immune homeostasis including tolerance. RA has been shown to enhance the in vitro differentiation of Treg cells that suppress immunity. Additionally, RA can suppress the activities of memory T cells, including decreasing the production of IFN-γ and increasing the secretion of IL-4, thereby promoting the differentiation of Treg cells. RA can also directly impair the differentiation pro-inflammatory Th17 cells that have been implicated in the development of many human autoimmune disorders. Lastly, RA can imprint leukocytes in general to change their homing characteristics to migrate to gut mucosa. Cells within the intestines produce RA, which can enhance Teff differentiation and inhibit pro-inflammatory Teff differentiation, support inducible Treg (iTreg) generation and influence Teff and iTreg expansion, gut homing and stability. Based on these activities, it was hypothesized that RAR agonists that could selectively effect the process that regulates the differentiation of Th17 and Treg cells could be effective therapeutic compounds because these RAR agonists could restore immune homeostasis initially disrupted by the underlying etiology of inflammation or an autoimmune disorder.

There are two main types of receptors that are required to mediate the effects of RA in mammals (and other organisms), the Retinoic Acid Receptors (RARs) and the Retinoid X Receptors (RXRs). Within each type there are three subtypes designated RAR alpha, RAR beta, and RAR gamma for the RAR family and RXR alpha, RXR beta, and RXR gamma for the RXR family. These receptor types are evolutionarily related but are functionally distinct. Each RAR subtype can bind to one of the RXR subtypes. The resulting RAR-RXR heterodimers interact with retinoic acid-response elements (RAREs) within the promoter regions of RA-inducible genes and function as activating transcription factors upon agonist binding to the RAR moiety of the heterodimer. The RAR-RXR heterodimers are not activated by ligands that bind to the RXR moiety of the heterodimer. RXR agonists, although they do not activate transcription through non-permissive heterodimers such as RAR-RXR heterodimers, regulate gene transcription through RXR-RXR homodimers or permissive heterodimers such as Nurr1-RXR, PPAR-RXR, LXR-RXR, and FXR-RXR.

Interestingly, RA signaling appears to have different temporal effects on T cell-mediated responses. For example, it seems that RA signaling is involved in an early T cell-mediated response that promotes a pro-immunity and/or pro-inflammatory response that reduces Treg cell differentiation and/or increases TH17 cell differentiation. Subsequently, however, RA signaling appears to be involved in a late T cell-mediated response that prevents a pro-immunity and/or pro-inflammatory response, by increasing Treg cell differentiation and/or reducing TH17 cell differentiation. Thus, inhibiting RA signaling during the early T cell-mediated response, but not late response, would be beneficial in treating a GVHD disorder. Also, RXR agonists promote Treg cell differentiation and concurrently Th17 cell formation by a RXR-mediated pathway that does not involve RA signaling. Thus, it could be of further benefit in treating a GVHD disorder to inhibit RA signaling during the early T cell-mediated response while concurrently or subsequently activating RXR signaling.

The present specification discloses compounds, compositions, and methods for treating an individual suffering from a GVHD disorder. This is accomplished by administering a therapeutically effective amount of a RAR antagonist or composition comprising such antagonist to an individual suffering from a GVHD disorder.

### SUMMARY

Thus, aspects of the present specification disclose a RAR antagonist. Non-limiting examples of a RAR antagonist are described in U.S. Pat. No. 5,952,345 and U.S. Pat. No. 5,958,954, the content of each of which is hereby incorporated by reference in its entirety. A RAR antagonist includes, without limitation, a RARα antagonist and a RARγ antagonist.

Other aspects of the present specification disclose a method of treating a transplant rejection, the method comprising the step of administering to an individual in need thereof a therapeutically effective amount of a RAR antagonist, wherein administration of the RAR antagonist reduces a symptom associated with the transplant rejection, thereby treating the individual. Aspects of the present specification also disclose a use of a RAR antagonist to treat a transplant rejection, wherein administration of the compound or composition reduces a symptom associated with the transplant rejection, thereby treating the individual.

Yet other aspects of the present specification disclose a method of treating a GVHD disorder, the method comprising the step of administering to an individual in need thereof a therapeutically effective amount of a RAR antagonist, wherein administration of the RAR antagonist reduces a symptom associated with the GVHD disorder, thereby treating the individual. Aspects of the present specification also disclose a use of a RAR antagonist to treat a GVHD disorder, wherein administration of the compound or composition reduces a symptom associated with the GVHD disorder, thereby treating the individual. Non-limiting examples of a symptom reduced by a method of treating a GVHD disorder disclosed herein include inflammation, fatigue, dizziness, malaise, elevated fever and high body temperature, extreme sensitivity to cold in the hands and feet, weakness and stiffness in muscles and joints, weight changes, digestive or gastrointestinal problems, low or high blood pressure, irritability, anxiety, or depression, infertility or reduced sex drive (low libido), blood sugar changes, and depending on the type of autoimmune disease, an increase in the size of an organ or tissue, or the destruction of an organ or tissue. Non-limiting examples of an inflammation symptom reduced by a method of treating a GVHD disorder include edema, hyperemia, erythema, bruising, tenderness, stiffness, swollenness, fever, a chill, congestion of the respiratory tract including nose, and bronchi, congestion of a sinus, a breathing problem, fluid retention, a blood clot, a loss of appetite, an increased heart rate, a formation of granulomas, fibrinous, pus, or non-viscous serous fluid, a formation of an ulcer, or pain.

Further aspects of the present specification disclose a method of promoting Treg cell differentiation in an individual, the method comprising the step of administering to the individual in need thereof a therapeutically effective amount of a RAR antagonist, wherein administration of the RAR antagonist promotes Treg cell differentiation. Aspects of the present specification also disclose a use of a RAR antagonist to promote Treg cell differentiation in an individual, wherein administration of the RAR antagonist to the individual promotes Treg cell differentiation. Administration of the RAR antagonist to the individual can also inhibit Th17 cell differentiation.

Further aspects of the present specification disclose a method of inhibiting Th17 cell differentiation in an individual, the method comprising the step of administering to the individual in need thereof a therapeutically effective amount of a RAR antagonist, wherein administration of the RAR antagonist inhibits Th17 cell differentiation. Aspects of the present specification also disclose a use of a RAR antagonist to inhibit Th17 cell differentiation in an individual, wherein administration of the RAR antagonist to the individual inhibits Th17 cell differentiation. Administration of the RAR antagonist to the individual can also promote Treg cell differentiation.

Other aspects of the present specification disclose a method of concurrently promoting Treg cell differentiation as well as inhibiting Th17 cell differentiation in an individual, the method comprising the step of administering to the individual in need thereof a therapeutically effective amount of a RAR antagonist, wherein administration of the RAR antagonist promotes Treg cell differentiation and inhibits Th17 cell differentiation. Aspects of the present specification also disclose a use of a RAR antagonist to concurrently promote Treg cell differentiation as well as inhibit Th17 cell differentiation in an individual, wherein administration of the RAR antagonist to the individual promotes Treg cell differentiation and inhibits Th17 cell differentiation.

Non-limiting examples of a RAR antagonist include a compound or a composition disclosed herein. Non-limiting examples of a transplant rejection include a hyperacute rejection, an acute rejection, or a chronic rejection, as well as, a graft-versus-host-disease. Non-limiting examples of a symptom reduced by a method of treating a transplant rejection disclosed herein include inflammation, fatigue, dizziness, malaise, elevated fever and high body temperature, extreme sensitivity to cold in the hands and feet, weakness and stiffness in muscles and joints, weight changes, digestive or gastrointestinal problems, low or high blood pressure, irritability, anxiety, or depression, infertility or reduced sex drive (low libido), blood sugar changes, and depending on the type of autoimmune disease, an increase in the size of an organ or tissue, or the destruction of an organ or tissue. Non-limiting examples of an inflammation symptom reduced by a method of treating a transplant rejection include edema, hyperemia, erythema, bruising, tenderness, stiffness, swollenness, fever, a chill, congestion of the respiratory tract including nose, and bronchi, congestion of a sinus, a breathing problem, fluid retention, a blood clot, a loss of appetite, an increased heart rate, a formation of granulomas, fibrinous, pus, or non-viscous serous fluid, a formation of an ulcer, or pain.

### BRIEF DESCRIPTION OF DRAWINGS

FIG. 1. Vitamin A quantification by B16-DR5 assay in vitro. Retinoic acid (RA) concentrations were quantified using the B16-DR5 assay. RA was titrated in serial dilutions (5 nM, 1 nM, 500 pM, 100 pM, 30 pM, and 0) (n=4/group). RA concentrations ≧30 pM were significantly higher than media alone. The findings are representative of three experiments.

FIG. 2A-E. Vitamin A metabolism is upregulated during acute GVHD. (FIG. 2A) Lethally irradiated B10.BR recipients injected with 10⁷ bone marrow cells (BM) and 15×10⁶ splenocytes from fully MHC-mismatched B6 mice. Day 7 and day 14 after bone marrow transplantation (BMT), tissues from lung, liver, small intestine, and colon were harvested and analyzed by B16-DR5 assay (n=4/group). The fold change was calculated by dividing the value of the BM only group or the GVHD group by the value of naïve mice group. (FIG. 2B) Lethally irradiated B10.BR recipients injected with 10⁷ wt B6 BM and 3×10⁶ purified RARE-luc B6 T cells. Day 7 and day 21 after BMT, tissues from liver, small intestine, and colon were harvested and analyzed by LC/MS-MS assay (n=4/group). (FIG. 2C) Lethally irradiated B10.BR recipients were injected with 10⁷ BM and 15×10⁶ B6 splenocytes from wild type (wt) B6 mice. Recipient mice (n=4/group) were sacrificed on day 14, along with 4 naïve B10.BR mice for control, and sections from frozen tissue blocks were analyzed for expression of CD11c⁺ and RALDH1 or RALDH2. (FIG. 2D) Lethally irradiated B10.BR recipients injected with 10⁷ BM and 15×10⁶ splenocytes from fully MHC-mismatched B6 mice. Lamina propria lymphocytes (LPLs) from the small intestine and lymphocytes in mesenteric lymph nodes (MLN) were isolated on day 14 and donor (H2k^{b}) CD11 c⁺ and CD11b⁺ cells were evaluated for aldehyde dehydrogenase (ALDH) enzymatic activity by ALDEFLUOR™ mean fluorescent intensity (MFI) (n=4/group). (FIG. 2E) Lethally irradiated B10.BR recipients were transplanted with 10⁷ BM and 3×10⁶ purified RARE-luc B6 T cells. RARE-luc T cells were quantified by emitted photons over total body area and within individual organs at serial time points after BMT (n=3-4/group). Fluorescence was detected with an Olympus FluoView 500 confocal laser scanning microscope. Original magnifications of 200× (RALDH1) and 400× (RALDH2).

FIG. 3. Heightened RA levels and RAR-α signaling exacerbates GVHD. Survival and weight curves of lethally irradiated B10.BR recipients (n=8/group) injected with 10⁷ BM and 15×10⁶ splenocytes from B6 mice. Subgroups were treated with vehicle (filled circles) or all trans retinoic acid (ATRA) (open circles) p=0.013.

FIG. 4. Heightened RA levels and RAR-α signaling exacerbates GVHD. Survival and weight curves of lethally irradiated B10.BR recipients (n=8/group) injected with 10⁷ BM and 5×10⁶ splenocytes from B6 mice. Subgroups were treated with vehicle (filled circles) or ATRA (open circles) p=0.037.

FIG. 5A-D. Inhibition of RAR-α signaling in donor T cells prevents GVHD lethality. (FIG. 5A) Survival and weight curves of lethally irradiated B10.BR recipients of 10⁷ BM and 10⁷ splenocytes from B6 mice. Recipients of splenocytes from dnRARα-CD4^{Cre} donors (filled circles) survived to a significantly greater extent than dnRARα donors (open circles) (p<0.001) (n=16/group). (FIG. 5B) Survival, weight, and clinical GVHD scores curves of lethally irradiated BALB/c recipients of 10⁷ BM and 5×10⁶ splenocytes from B6 mice. Recipients of splenocytes from dnRARα-CD4^{Cre} donors (filled circles) survived to a significantly greater extent than dnRARα donors (open circles) (p<0.001) (n=13-14/group). (FIG. 5C) Tissues (lung, liver, spleen, ileum and colon) from B10.BR recipients or BALB/c recipients were harvested at day 21 after transplant, stained with hematoxylin and eosin (H&E) and scored for GVHD (means±SE) (n=4-5/group). (FIG. 5D) FITC-dextran was administered orally to BALB/c recipients on day 21. Serum levels were measured 4 hours later. Indicate recipients of wt BM if not otherwise indicated (P=0.007). *p>0.05, **p>0.01, and ***p>0.001.

FIG. 6. Blocked RAR-α signaling on donor T cells does not alter T cell proliferation in vivo. 20×10⁷ CFSE-labeled dnRARα, or dnRARα-CD4^{Cre} splenocytes were transferred into lethally irradiated BALB/c recipients. Spleens were harvested and analyzed by flow cytometry for carboxyfluorescein succinimidyl ester (CFSE) dilution on day 4. Cells were gated on H-2K^{b} positive events. (n=4 mice/group).

FIG. 7A-C. Donor T cells blocked RAR-α signaling impairs the integrin and chemokine expression and skews T cell polarity toward the Th2 phenotype. (FIGS. 7A-C) Lethally irradiated BALB/c recipients (n=4-5/group) were transplanted with 10⁷ BM from B6 mice and 1.5x0⁶ dnRARα (open circles) or dnRARα-CD4^{Cre} (filled circles) purified T cells. Splenocytes, liver cells, and colon LPLs were isolated on indicated days and analyzed by FACS. Cells were gated on H-2K^{b} positive events. (FIG. 7A) The frequency of CD4⁺ cells expressing IFN-γ, IL-4, and IL-17 from spleen and colon LPLs is shown. (FIG. 7B) The absolute number of CD4⁺ cells expressing CXCR3, α4β7, and CCR9 in spleen, MLN, and liver cells is depicted. (FIG. 7C) The absolute number of total, CD4⁺ cells, and CD8⁺ cells and the absolute number of CD4⁺ and CD8⁺ cells expressing CXCR3, α4β7, and CCR9 in colon LPLs are shown. *p<0.05, **p<0.01, and ***p<0.001.

FIG. 8. Donor CD8⁺ T cells with inhibited RA signaling have impaired intestinal homing chemokine and integrin expression during GVHD. Lethally irradiated BALB/c recipients (n=4=5/group) were transplanted with 10⁷ B6 BM and 1.5×10⁶ dnRARα (open circles) or dnRARα-CD4^{Cre} (filled circles) purified T cells. Splenocytes, MLN, and liver cells, were isolated on day 6 post-BMT and analyzed by FACS. Cells were gated on H-2K^{b} positive events. The absolute number of CD8⁺ cells expressing CXCR3, α4β7, and CCR9 in spleen, MLN, and liver cells is depicted. *p<0.05, **p<0.01, and ***p<0.001.

FIG. 9A-B. Donor T cells RAR-α signaling is essential for the integrin and chemokine expression and skews T cell polarity toward the Th1 phenotype independent on donor-derived T cell cytokines. Lethally irradiated BALB/c recipients (n=4-5/group) were transplanted with 10⁷ CD45.2⁺ B6 TCD-BM (T cell depleted bone marrow), with 1×10⁶ CD45.1⁺ T cells and 1×10⁶ CD45.2⁺ T cells from dnRARα (CD45.1 with dnRARα and dnRARα with CD45.1), dnRARα-CD4^{Cre} (CD45.1 with dnRARα-CD4^{Cre} and dnRARα-CD4^{Cre} with CD45.1), or dnRARα-CD4^{Cre} purified T cells only. Lymphocytes from spleens were isolated on day 7 and analyzed by FACS. The absolute number of CD4⁺ (FIG. 9A) and CD8⁺ (FIG. 9B) cells expressing CXCR3 and α4β7 are depicted. *p<0.05, **p<0.01, and ***p<0.001.

FIG. 10A-B. RAR-α signaling in donor T cells is essential for acute GVHD lethality independent on conditioning. (FIG. 10A) Unirradiated B6D2F1 (n=5-11/group) were transplanted with 12×10⁷ splenocytes from syngeneic, dnRARα (open circles), or dnRARα-CD4^{Cre} (filled circles) mice on day 0. Survival, weight, and clinical GVHD scores curves are shown. (p<0.001). (FIG. 10B) In a separate study, unirradiated or irradiated (12Gy; radiation therapy, RT) B6D2F1 (n=5/group) were transplanted with 12×10⁷ splenocytes (unirradiated mice) or 5×10⁶ splenocytes (irradiated mice) from dnRARα-CD4^{Cre} or dnRARα mice on day 0. The frequency of CD4⁺ and CD8⁺ cells expressing CXCR3, α4β7, and CCR9 were analyzed in the peripheral blood on day 7. *p<0.05, **p<0.01, and ***p<0.001.

FIG. 11A-E. Inhibition of RAR-α signaling in donor T cells increases Tregs in vivo. Lethally irradiated BALB/c recipients (n=4-8/group) were transplanted with 10⁷ B6 BM and 1.5×10⁶ dnRARα (open circles), or dnRARα-CD4^{Cre} (filled circles) purified T cells. (FIG. 11A) The frequency of CD4⁺ Foxp3⁺ cells and the absolute number of CD4⁺ Foxp3⁺ cells in the spleen are shown. (FIG. 11B) Liver cells and colon LPLs were isolated on day 14 and analyzed by FACS. The frequency of CD4⁺ Foxp3⁺ cells is shown. (FIG. 11C) Lethally irradiated BALB/c recipients were transplanted with 10⁷ CD45.2⁺ TCD B6 BM and 1×10⁶ CD45.1⁺ B6 T cells and 1×10⁶ CD45.2⁺ T cells from either dnRARα (CD45.1 with dnRARα), dnRARαCD4^{Cre} (CD45.1 with dnRARα-CD4^{Cre}) mice, or transplanted 10⁷ CD45.2⁺ TCD-B6 BM and 2×10⁶ dnRARα-CD4^{Cre} T cells alone. The percentages of chimerism of CD4⁺ cells and the frequency of CD4⁺ Foxp3⁺ cells gated on CD45.2⁺ (dnRARα or dnRARαCD4^{Cre}) or CD45.1⁺ in spleens on day14 after BMT are shown. (FIG. 11D) Lethally irradiated BALB/c recipients (n=7-8/group) were injected with 10⁷ CD45.1⁺ TCD-BM with and 1×10⁶ CD25 depleted T cells from dnRARα-CD4^{Cre} mice or 10⁷ CD45.1⁺ TCD-BM with CD25repleted T cells from either dnRARα or dnRARα-CD4^{Cre} mice. The frequency of CD4⁺ Foxp3⁺ cells gated on CD45.2⁺ (T cell origin) or CD45.1⁺ (donor BM origin) in spleens on day14 after BMT are shown. (FIG. 11E) Lethally irradiated BALB/c recipients (n=8/group) were injected with 10⁷ BM and 5×10⁶ CD25-depleted splenocytes or CD25-replete splenocytes from either dnRARα or dnRARα-CD4^{Cre} and monitored for survival. *p<0.05, **p<0.01, and ***p<0.001.

FIG. 12A-E. Blocked RAR signaling on donor T cells dose not abort GvL effect. Lethally irradiated BALB/c recipients were transplanted with 10⁷ T cell-depleted BM with or without 3×10⁵ A20 lymphoma cells on day 0. Subgroups were transplanted with 5×10⁶ splenocytes from dnRARα (open circles) and dnRARα-CD4^{Cre} (filled triangles) also on day 0. (FIGS. 12A-C) Survival, weight, clinical GVHD scores of lethally irradiated BALB/c recipients (n=5-10/group) transplanted with BM only (filled squares), BM+A20 or together with A20+5×10⁶ splenocytes from dnRARα (open circles) and dnRARα-CD4^{Cre} (filled triangles); (FIG. 12D) Tumor growth was monitored by luciferase imaging on week 1 to 4, 6, 8, and 15 after BMT. (FIG. 12E) The frequency of CD8⁺ cells expressing granzyme B was analyzed in the spleen on day 14. *p<0.05, **p<0.01, and ***p<0.001.

FIG. 13. Donor T cells blocked RAR-α signaling does not impair TNF-α production in donor T cells. The frequency of CD4⁺ and CD8⁺ cells expressing TNF-α were analyzed isolated from the splenocytes and liver cells on day 6. n=4/group. p=ns

### DESCRIPTION

Disclosed herein are methods of treating a graft-versus-host disease (GVHD) disorder by administering an RAR antagonist.

Thus, aspects of the present specification provide, in part, a RAR antagonist. As used herein, the term “RAR antagonist” refers to a compound that selectively binds to one or more RAR receptors like a RARα, a RARβ, or a RARγ in a manner that reduces or prevents gene transcription via an RAR response element. As used herein, the term “selectively binds,” when made in reference to a RAR antagonist, refers to the discriminatory binding of a RAR antagonist to the indicated target receptor. For example, a RARα antagonist will discriminatorily bind to a RARα, but does not substantially bind with non-target receptors like a RARβ and/or a RARγ. Similarly, a RARγ antagonist will discriminatorily bind to a RARγ, but does not substantially bind with non-target receptors like a RARβ and/or a RARα.

Selective binding of a RARα antagonist to a RARα includes binding properties such as, e.g., binding affinity and binding specificity. Binding affinity refers to the length of time a RARα antagonist resides at its a RARα binding site, and can be viewed as the strength with which a RARα antagonist binds its a RARα. Binding affinity can be described a RARα agonist's equilibrium dissociation constant (KD), which is defined as the ratio Kd/Ka at equillibrium. Where Ka is a RARα agonist's association rate constant and kd is a RARα agonist's dissociation rate constant. Binding affinity is determined by both the association and the dissociation and alone neither high association nor low dissociation can ensure high affinity. The association rate constant (Ka), or on-rate constant (Kon), measures the number of binding events per unit time, or the propensity of a RARα antagonist and its RARα to associate reversibly into its agonist-receptor complex. The association rate constant is expressed in M⁻¹ s⁻¹, and is symbolized as follows: [Ag]×[α]×Kon. The larger the association rate constant, the more rapidly a RARα antagonist binds to its RARα, or the higher the binding affinity between agonist and receptor. The dissociation rate constant (Kd), or off-rate constant (Koff), measures the number of dissociation events per unit time propensity of an agonist-receptor complex to separate (dissociate) reversibly into its component molecules, namely the RARα antagonist and the RARα. The dissociation rate constant is expressed in s⁻¹, and is symbolized as follows: [Ag+Rc]×Koff. The smaller the dissociation rate constant, the more tightly bound a RARα antagonist is to its RARα, or the higher the binding affinity between agonsit and receptor. The equilibrium dissociation constant (KD) measures the rate at which new agonist-receptor complexes formed equals the rate at which agonist-receptor complexes dissociate at equilibrium. The equilibrium dissociation constant is expressed in M, and is defined as Koff/Kon=[Ag]×[Rc]/[Ag+Rc], where [Ag] is the molar concentration of a RARα agonist, [α] is the molar concentration of the RARα, and [Ag+R^{c}] is the of molar concentration of the agonist-receptor complex, where all concentrations are of such components when the system is at equilibrium. The smaller the equilibrium dissociation constant, the more tightly bound a RARα antagonist is to its RARα, or the higher the binding affinity between antagonist and receptor.

In aspects of this embodiment, the binding affinity of a RARα antagonist that selectively binds to a RARα can have an association rate constant of, e.g., less than 1×10⁵ M⁻¹ s⁻¹, less than 1×10⁶ M⁻¹ less than 1×10⁷ M⁻¹ s⁻¹, or less than 1×10⁸ M⁻¹ s⁻¹. In another embodiment, the binding affinity of a RARα antagonist that selectively binds to a RARα can have an association rate constant of, e.g., more than 1×10⁵ M⁻¹ s⁻¹, more than 1×10⁶ M⁻¹ s⁻¹, more than 1×10⁷ M⁻¹ s⁻¹, or more than 1×10⁸ M⁻¹ s⁻¹. In other aspects, the binding affinity of a RARα antagonist that selectively binds to a RARα can have an association rate constant between, e.g., 1×10⁵ M⁻¹ s⁻¹ to 1×10⁸ M⁻¹ S⁻¹, 1×10⁶ M⁻¹ s⁻¹ to 1×10⁸ M⁻¹ s⁻¹, 1×10⁵ M⁻¹ s⁻¹ to 1×10⁷ M⁻¹ s⁻¹, or 1×10⁶ M⁻¹ s⁻¹ to 1×10⁷ M⁻¹ s⁻¹.

In other aspects of this embodiment, the binding affinity of a RARα antagonist that selectively binds to a RARα can have a disassociation rate constant of, e.g., less than 1×10⁻³ s⁻¹, less than 1×10⁻⁴ s⁻¹, or less than 1×10⁻⁵ s⁻¹. In another embodiment, the binding affinity of a RARα antagonist that selectively binds to a RARα can have a disassociation rate constant of, e.g., more than 1×10⁻³ s⁻¹, more than 1×10⁻⁴ s⁻¹, or more than 1×10⁻⁵ s⁻¹. In other aspects, the binding affinity of a RARα antagonist that selectively binds to a RARα can have a disassociation rate constant between, e.g., 1×10⁻³ s⁻¹ to 1×10⁻⁵ s⁻¹, 1×10⁻³ s⁻¹ to 1×10⁻⁴ s⁻¹, or 1×10⁴ s⁻¹ to 1×10⁻⁵ s⁻¹.

In yet other aspects of this embodiment, the binding affinity of a RARα antagonist that selectively binds to a RARα can have an equilibrium disassociation constant of less than 100 nM. In aspects of this embodiment, the binding affinity of a RARα antagonist that selectively binds to a RARα can have an equilibrium disassociation constant of, e.g., less than 100 nM, less than 90 nM, less than 80 nM, less than 70 nM, less than 60 nM, less than 50 nM, less than 40 nM, less than 30 nM, less than 20 nM, or less than 10 nM. In aspects of this embodiment, the binding affinity of a RARα antagonist that selectively binds to a RARα can have an equilibrium disassociation between, e.g., 0.1 nM to 10 nM, 0.1 nM to 50 nM, 0.1 nM to 100 nM, 0.5 nM to 10 nM, 0.5 nM to 50 nM, 0.5 nM to 100 nM, 1 nM to 10 nM, 1 nM to 50 nM, or 1 nM to 100 nM.

In still other aspects of this embodiment, the binding affinity of a RARα antagonist that selectively binds to a RARα can have an association rate constant for a RARβ, or a RARγ of, e.g., less than 1×10⁰ M⁻¹ s⁻¹, less than 1×10¹ M⁻¹ s⁻¹, less than 1×10² M⁻¹ s⁻¹, less than 1×10³ M⁻¹ s⁻¹, or less than 1×10⁴ M⁻¹ s⁻¹. In another embodiment, the binding affinity of a RARα antagonist that selectively binds to a RARα can have an association rate constant of a RARβ, or a RARγ of, e.g., at most 1×10⁰ M⁻¹ s⁻¹, at most 1×10⁻¹ M⁻¹ s⁻¹, at most 1×10² M⁻¹ s⁻¹, at most 1×10³ M⁻¹ s⁻¹, or at most 1×10⁴ M⁻¹ s⁻¹.

In further aspects of this embodiment, the binding affinity of a RARα antagonist that selectively binds to a RARα can have an equilibrium disassociation constant for a RARβ or a RARγ of, e.g., more than 500 nM, for than 1,000 nM, more than 5,000 nm, or more than 10,000 nM. In another embodiment, the binding affinity of a RARα antagonist that selectively binds to a RARα can have an equilibrium disassociation constant for a RARβ or a RARγ between, e.g., 500 nM to 10,000 nM, 1,000 nM to 10,000 nM, or 5,000 nM to 10,000 nM.

Binding specificity is the ability of a RARα antagonist to discriminate between a RARα and a receptor that does not contain its binding site, such as, e.g., a RARβ or a RARγ. One way to measure binding specificity is to compare the Kon association rate of a RARα antagonist for its RARα relative to the Kon association rate of a RARα antagonist for a receptor that does not contain its binding site. For example, comparing the association rate constant (Ka) of a RARα antagonist for its RARα relative to a RARβ and/or a RARγ.

In aspects of this embodiment, a RARα antagonist that selectively binds to a RARα can have an association rate constant (Ka) for a receptor not comprising its binding site of, e.g., less than 1×10⁰ M⁻¹ s⁻¹ less than 1×10¹ M⁻¹ s⁻¹ less than 1×10² M⁻¹ s⁻¹, less than 1×10³ M⁻¹ s⁻¹ or less than 1×10⁴ M⁻¹ s⁻¹. In other aspects of this embodiment, a RARα antagonist that selectively binds to a RARα can have an association rate constant (Ka) for a receptor not comprising its binding site of, e.g., at most 1×10⁰ M⁻¹ s⁻¹ at most 1×10¹ M⁻¹ s⁻¹, at most 1×10² M⁻¹ s⁻¹, at most 1×10³ M⁻¹ s⁻¹ or at most 1×10⁴ M⁻¹ s⁻¹.

In other aspects of this embodiment, a RARα antagonist that selectively binds to a RARα can have an association rate constant (Ka) for a receptor not comprising its binding site of, e.g., at least 2-fold more, at least 3-fold more, at least 4-fold more, at least 5-fold more, at least 6-fold more, at least 7-fold more, at least 8-fold more, or at least 9-fold more. In further aspects of this embodiment, a RARα antagonist that selectively binds to a RARα can have an association rate constant (Ka) for a receptor not comprising its binding site of, e.g., at least 10-fold more, at least 100-fold more, at least 1.000-fold more or at least 10.000-fold more. In yet other aspects of this embodiment, a RARα antagonist that selectively binds to a RARα can have an association rate constant (Ka) for a receptor not comprising its binding site of, e.g., at most 1-fold more, at most 2-fold more, at most 3-fold more, at most 4-fold more, at most 5-fold more, at most 6-fold more, at most 7-fold more, at most 8-fold more, or at most 9-fold more. In yet other aspects of this embodiment, a RARα antagonist that selectively binds to a RARα can have an association rate constant (Ka) for a receptor not comprising its binding site of, e.g., at most 10-fold more, at most 100-fold more, at most 1.000-fold more or at most 10.000-fold more.

The binding specificity of a RARα antagonist that selectively binds to a RARα can also be characterized as a binding ratio that such a RARα antagonist can discriminate its RARα relative to a receptor not comprising its binding site, such as, e.g., a RARβ or a RARγ. In aspects of this embodiment, a RARα antagonist that selectively binds to a RARα has a binding ratio for its RARα relative to a receptor not comprising its binding site of, e.g., at least 2:1, at least 3:1, at least 4:1, at least 5:1, at least 64:1, at least 7:1, at least 8:1, at least 9:1, at least 10:1, at least 15:1, at least 20:1, at least 25:1, at least 30:1, at least 35:1, or at least 40:1. In other aspects of this embodiment, a RARα antagonist that selectively binds to a RARα has a binding ratio for its RARα relative to a RARβ and/or a RARγ of, e.g., at least 2:1, at least 3:1, at least 4:1, at least 5:1, at least 64:1, at least 7:1, at least 8:1, at least 9:1, at least 10:1, at least 15:1, at least 20:1, at least 25:1, at least 30:1, at least 35:1, or at least 40:1.

In aspects of this embodiment, a RARα antagonist will have a ratio of activity at a RARα relative to a RARβ and/or a RARγ of, e.g., at least 5 greater, at least 10 greater, at least 15, or at least 20 greater. A RAR pan agonist will have activity at a RARα, a RARβ, and a RARγ, i.e., similar potency at a RARα, a RARβ, and a RARγ.

The binding specificity of a RARα antagonist that selectively binds to a RARα can also be characterized as an activity ratio that such a RARα antagonist can exert activity through binding to its RARα relative to a receptor not comprising its binding site, such as, e.g., a RARβ or a RARγ. In aspects of this embodiment, a RARα antagonist that selectively binds to a RARα has an activity ratio through its RARα relative to a receptor not comprising its binding site of, e.g., at least 2:1, at least 3:1, at least 4:1, at least 5:1, at least 64:1, at least 7:1, at least 8:1, at least 9:1, at least 10:1, at least 15:1, at least 20:1, at least 25:1, at least 30:1, at least 35:1, or at least 40:1. In other aspects of this embodiment, a RARα antagonist that selectively binds to a RARα has an activity ratio through its RARα relative to a RARβ and/or a RARγ of, e.g., at least 2:1, at least 3:1, at least 4:1, at least 5:1, at least 64:1, at least 7:1, at least 8:1, at least 9:1, at least 10:1, at least 15:1, at least 20:1, at least 25:1, at least 30:1, at least 35:1, or at least 40:1.

Selective binding of a RARγ antagonist to a RARγ includes binding properties such as, e.g., binding affinity and binding specificity. Binding affinity refers to the length of time a RARγ antagonist resides at its a RARγ binding site, and can be viewed as the strength with which a RARγ antagonist binds its a RARγ. Binding affinity can be described a RARγ agonist's equilibrium dissociation constant (KD), which is defined as the ratio Kd/Ka at equillibrium. Where Ka is a RARγ agonist's association rate constant and kd is a RARγ agonist's dissociation rate constant. Binding affinity is determined by both the association and the dissociation and alone neither high association nor low dissociation can ensure high affinity. The association rate constant (Ka), or on-rate constant (Kon), measures the number of binding events per unit time, or the propensity of a RARγ antagonist and its RARγ to associate reversibly into its agonist-receptor complex. The association rate constant is expressed in M⁻¹ s⁻¹, and is symbolized as follows: [Ag]×[α]×Kon. The larger the association rate constant, the more rapidly a RARγ antagonist binds to its RARγ, or the higher the binding affinity between agonist and receptor. The dissociation rate constant (Kd), or off-rate constant (Koff), measures the number of dissociation events per unit time propensity of an agonist-receptor complex to separate (dissociate) reversibly into its component molecules, namely the RARγ antagonist and the RARγ. The dissociation rate constant is expressed in s⁻¹, and is symbolized as follows: [Ag+Rc]×Koff. The smaller the dissociation rate constant, the more tightly bound a RARγ antagonist is to its RARγ, or the higher the binding affinity between agonsit and receptor. The equilibrium dissociation constant (KD) measures the rate at which new agonist-receptor complexes formed equals the rate at which agonist-receptor complexes dissociate at equilibrium. The equilibrium dissociation constant is expressed in M, and is defined as Koff/Kon=[Ag]×[Rc]/[Ag+Rc], where [Ag] is the molar concentration of a RARγ agonist, [Rc] is the molar concentration of the RARγ, and [Ag+Rc] is the of molar concentration of the agonist-receptor complex, where all concentrations are of such components when the system is at equilibrium. The smaller the equilibrium dissociation constant, the more tightly bound a RARγ antagonist is to its RARγ, or the higher the binding affinity between agonist and receptor.

In aspects of this embodiment, the binding affinity of a RARγ antagonist that selectively binds to a RARγ can have an association rate constant of, e.g., less than 1×10⁵ M⁻¹ s⁻¹, less than 1×10⁶ M⁻¹ less than 1×10⁷ M⁻¹ s⁻¹, or less than 1×10⁸ M⁻¹ s⁻¹. In another embodiment, the binding affinity of a RARγ antagonist that selectively binds to a RARγ can have an association rate constant of, e.g., more than 1×10⁵ M⁻¹ s⁻¹, more than 1×10⁶ M⁻¹ s⁻¹, more than 1×10⁷ M⁻¹ s⁻¹, or more than 1×10⁸ M⁻¹ s⁻¹. In other aspects, the binding affinity of a RARγ antagonist that selectively binds to a RARγ can have an association rate constant between, e.g., 1×10⁵ M⁻¹ s⁻¹ to 1×10⁸ M⁻¹ s⁻¹, 1×10⁶ M⁻¹ s⁻¹ to 1×10⁸ M⁻¹ s⁻¹, 1×10⁵ M⁻¹ s⁻¹ to 1×10⁷ M⁻¹ s⁻¹, or 1×10⁶ s⁻¹ to 1×10⁷ M⁻¹ s⁻¹.

In other aspects of this embodiment, the binding affinity of a RARγ antagonist that selectively binds to a RARγ can have a disassociation rate constant of, e.g., less than 1×10⁻³ s⁻¹, less than 1×10⁻⁴ s⁻¹, or less than 1×10⁻⁵ s⁻¹. In another embodiment, the binding affinity of a RARγ antagonist that selectively binds to a RARγ can have a disassociation rate constant of, e.g., more than 1×10⁻³ s⁻¹, more than 1×10⁻⁴ s⁻¹, or more than 1×10⁻⁵ s⁻¹. In other aspects, the binding affinity of a RARγ antagonist that selectively binds to a RARγ can have a disassociation rate constant between, e.g., 1×10⁻³ s⁻¹ to 1×10⁻⁵ s⁻¹, 1×10⁻³ s⁻¹ to 1×10⁻⁴ s⁻¹, or 1×10⁴ s⁻¹ to 1×10⁻⁵ s⁻¹.

In yet other aspects of this embodiment, the binding affinity of a RARγ antagonist that selectively binds to a RARγ can have an equilibrium disassociation constant of less than 100 nM. In aspects of this embodiment, the binding affinity of a RARγ antagonist that selectively binds to a RARγ can have an equilibrium disassociation constant of, e.g., less than 100 nM, less than 90 nM, less than 80 nM, less than 70 nM, less than 60 nM, less than 50 nM, less than 40 nM, less than 30 nM, less than 20 nM, or less than 10 nM. In aspects of this embodiment, the binding affinity of a RARγ antagonist that selectively binds to a RARγ can have an equilibrium disassociation between, e.g., 0.1 nM to 10 nM, 0.1 nM to 50 nM, 0.1 nM to 100 nM, 0.5 nM to 10 nM, 0.5 nM to 50 nM, 0.5 nM to 100 nM, 1 nM to 10 nM, 1 nM to 50 nM, or 1 nM to 100 nM.

In still other aspects of this embodiment, the binding affinity of a RARγ antagonist that selectively binds to a RARγ can have an association rate constant for a RARβ, or a RARγ of, e.g., less than 1×10⁰ M⁻¹ s⁻¹, less than 1×10¹ M⁻¹ s⁻¹, less than 1×10² M⁻¹ s⁻¹, less than 1×10³ M⁻¹ s⁻¹, or less than 1×10⁴ M⁻¹ s⁻¹. In another embodiment, the binding affinity of a RARγ antagonist that selectively binds to a RARγ can have an association rate constant of a RARβ, or a RARγ of, e.g., at most 1×10⁰ M⁻¹ s⁻¹ at most 1×10¹ M⁻¹ s⁻¹ at most 1×10² M⁻¹ s⁻¹, at most 1×10³ M⁻¹ s⁻¹, or at most 1×10⁴ M⁻¹ s⁻¹.

In further aspects of this embodiment, the binding affinity of a RARγ antagonist that selectively binds to a RARγ can have an equilibrium disassociation constant for a RARα or a RARβ of, e.g., more than 500 nM, for than 1,000 nM, more than 5,000 nm, or more than 10,000 nM. In another embodiment, the binding affinity of a RARγ antagonist that selectively binds to a RARγ can have an equilibrium disassociation constant for a RARα or a RARβ between, e.g., 500 nM to 10,000 nM, 1,000 nM to 10,000 nM, or 5,000 nM to 10,000 nM.

Binding specificity is the ability of a RARγ antagonist to discriminate between a RARγ and a receptor that does not contain its binding site, such as, e.g., a RARα or a RARβ. One way to measure binding specificity is to compare the Kon association rate of a RARγ antagonist for its RARγ relative to the Kon association rate of a RARγ antagonist for a receptor that does not contain its binding site. For example, comparing the association rate constant (Ka) of a RARγ antagonist for its RARγ relative to a RARβ and/or a RARγ.

In aspects of this embodiment, a RARγ antagonist that selectively binds to a RARγ can have an association rate constant (Ka) for a receptor not comprising its binding site of, e.g., less than 1×10⁰ M⁻¹ s⁻¹ less than 1×10¹ M¹ s⁻¹ less than 1×10² M⁻¹ s⁻¹, less than 1×10³ M⁻¹ s⁻¹ or less than 1×10⁴ M⁻¹ s⁻¹. In other aspects of this embodiment, a RARγ antagonist that selectively binds to a RARγ can have an association rate constant (Ka) for a receptor not comprising its binding site of, e.g., at most 1×10⁰ M⁻¹ s⁻¹ at most 1×10¹ M⁻¹ s⁻¹, at most 1×10² M⁻¹ s⁻¹, at most 1×10³ M⁻¹ s⁻¹ or at most 1×10⁴ M⁻¹ s⁻¹.

In other aspects of this embodiment, a RARγ antagonist that selectively binds to a RARγ can have an association rate constant (Ka) for a receptor not comprising its binding site of, e.g., at least 2-fold more, at least 3-fold more, at least 4-fold more, at least 5-fold more, at least 6-fold more, at least 7-fold more, at least 8-fold more, or at least 9-fold more. In further aspects of this embodiment, a RARγ antagonist that selectively binds to a RARγ can have an association rate constant (Ka) for a receptor not comprising its binding site of, e.g., at least 10-fold more, at least 100-fold more, at least 1,000-fold more or at least 10,000-fold more. In yet other aspects of this embodiment, a RARγ antagonist that selectively binds to a RARγ can have an association rate constant (Ka) for a receptor not comprising its binding site of, e.g., at most 1-fold more, at most 2-fold more, at most 3-fold more, at most 4-fold more, at most 5-fold more, at most 6-fold more, at most 7-fold more, at most 8-fold more, or at most 9-fold more. In yet other aspects of this embodiment, a RARγ antagonist that selectively binds to a RARγ can have an association rate constant (Ka) for a receptor not comprising its binding site of, e.g., at most 10-fold more, at most 100-fold more, at most 1,000-fold more or at most 10,000-fold more.

The binding specificity of a RARγ antagonist that selectively binds to a RARγ can also be characterized as a binding ratio that such a RARγ antagonist can discriminate its RARγ relative to a receptor not comprising its binding site, such as, e.g., a RARα or a RARβ. In aspects of this embodiment, a RARγ antagonist that selectively binds to a RARγ has a binding ratio for its RARγ relative to a receptor not comprising its binding site of, e.g., at least 2:1, at least 3:1, at least 4:1, at least 5:1, at least 64:1, at least 7:1, at least 8:1, at least 9:1, at least 10:1, at least 15:1, at least 20:1, at least 25:1, at least 30:1, at least 35:1, or at least 40:1. In other aspects of this embodiment, a RARγ antagonist that selectively binds to a RARγ has a binding ratio for its RARγ relative to a RARβ and/or a RARγ of, e.g., at least 2:1, at least 3:1, at least 4:1, at least 5:1, at least 64:1, at least 7:1, at least 8:1, at least 9:1, at least 10:1, at least 15:1, at least 20:1, at least 25:1, at least 30:1, at least 35:1, or at least 40:1.

In aspects of this embodiment, a RARγ antagonist will have a ratio of activity at a RARγ relative to a RARβ and/or a RARγ of, e.g., at least 5 greater, at least 10 greater, at least 15, or at least 20 greater. A RAR pan agonist will have activity at a RARγ, a RARβ, and a RARγ, i.e., similar potency at a RARγ, a RARβ, and a RARγ.

The binding specificity of a RARγ antagonist that selectively binds to a RARγ can also be characterized as an activity ratio that such a RARγ antagonist can exert activity through binding to its RARγ relative to a receptor not comprising its binding site, such as, e.g., a RARα or a RARβ. In aspects of this embodiment, a RARγ antagonist that selectively binds to a RARγ has an activity ratio through its RARγ relative to a receptor not comprising its binding site of, e.g., at least 2:1, at least 3:1, at least 4:1, at least 5:1, at least 64:1, at least 7:1, at least 8:1, at least 9:1, at least 10:1, at least 15:1, at least 20:1, at least 25:1, at least 30:1, at least 35:1, or at least 40:1. In other aspects of this embodiment, a RARγ antagonist that selectively binds to a RARγ has an activity ratio through its RARγ relative to a RARβ and/or a RARγ of, e.g., at least 2:1, at least 3:1, at least 4:1, at least 5:1, at least 64:1, at least 7:1, at least 8:1, at least 9:1, at least 10:1, at least 15:1, at least 20:1, at least 25:1, at least 30:1, at least 35:1, or at least 40:1.

In an aspect of this embodiment, a RAR antagonist is a compound that selectively binds to one or more RAR receptors, such as the RAR antagonists disclosed in U.S. Pat. Nos. 5,952,345 and/or 5,958,954, the contents of each of which are incorporated by reference herein for all they disclose regarding RAR antagonists. In one embodiment, a RARα antagonist is a compound having the structure of formula I:

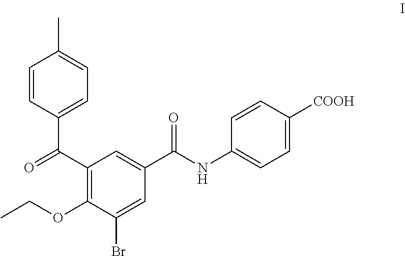 [SMILES: CCOc1c(Br)cc(C(=O)Nc2ccc(C(=O)O)cc2)cc1C(=O)c1ccc(C)cc1]

or a pharmaceutically acceptable salt thereof. In an aspect of this embodiment, a RARγ antagonist is a compound having the structure of formula II:

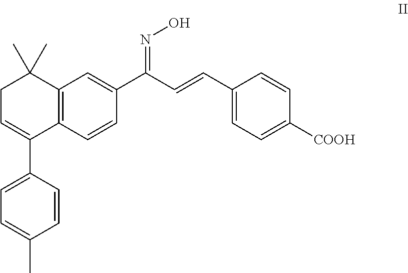 [SMILES: Cc1ccc(C2=CCC(C)(C)c3cc(C(/C=C/c4ccc(C(=O)O)cc4)=N/O)ccc32)cc1]

or a pharmaceutically acceptable salt thereof.

Aspects of the present specification provide, in part, a RAR antagonist having activity that promotes Treg cell differentiation. In aspects of this embodiment, a RAR antagonist promotes Treg cell differentiation by at least 10%, at least 20%, at least 30%, at least 40%, at least 50%, at least 60%, at least 70%, at least 80%, at least 90%, at least 100%, at least 200%, at least 300%, at least 400%, or at least 500%. In other aspects of this embodiment, a RAR antagonist promotes Treg cell differentiation by about 10% to about 25%, about 10% to about 50%, about 10% to about 75%, about 10% to about 100%, about 10% to about 200%, about 10% to about 300%, about 10% to about 400%, about 10% to about 500%, about 25% to about 50%, about 25% to about 75%, about 25% to about 100%, about 25% to about 200%, about 25% to about 300%, about 25% to about 400%, about 25% to about 500%, about 50% to about 100%, about 50% to about 200%, about 50% to about 300%, about 50% to about 400%, or about 50% to about 500%.

In an embodiment, a RAR antagonist has activity that results in increased Foxp3 expression in cells exposed to the RAR antagonist. In aspects of this embodiment, a RAR antagonist increases Foxp3 expression in cells by at least 10%, at least 20%, at least 30%, at least 40%, at least 50%, at least 60%, at least 70%, at least 80%, at least 90%, at least 100%, at least 200%, at least 300%, at least 400%, or at least 500%, relative to cells not exposed to the same RAR antagonist. In other aspects of this embodiment, a RAR antagonist increases Foxp3 expression in cells by about 10% to about 25%, about 10% to about 50%, about 10% to about 75%, about 10% to about 100%, about 10% to about 200%, about 10% to about 300%, about 10% to about 400%, about 10% to about 500%, about 25% to about 50%, about 25% to about 75%, about 25% to about 100%, about 25% to about 200%, about 25% to about 300%, about 25% to about 400%, about 25% to about 500%, about 50% to about 100%, about 50% to about 200%, about 50% to about 300%, about 50% to about 400%, or about 50% to about 500%, relative to cells not exposed to the same RAR antagonist.

In another aspect of this embodiment, a RAR antagonist has activity that results in increased Foxp3 expression in naïve CD4⁺ CD25⁻ FoxP3⁻ cells cultured under Treg cell differentiation conditions. In other aspects of this embodiment, a RAR antagonist increases Foxp3 expression in naïve CD4⁺ CD25⁻ FoxP3⁻ cells cultured under Treg cell differentiation by at least 10%, at least 20%, at least 30%, at least 40%, at least 50%, at least 60%, at least 70%, at least 80%, at least 90%, at least 100%, at least 200%, at least 300%, at least 400%, or at least 500%, relative to naïve CD4⁺ CD25⁻ FoxP3⁻ cells cultured under Treg cell differentiation not exposed to the same RAR antagonist. In yet other aspects of this embodiment, a RAR antagonist increases Foxp3 expression in naïve CD4⁺ CD25⁻ FoxP3⁻ cells cultured under Treg cell differentiation by about 10% to about 25%, about 10% to about 50%, about 10% to about 75%, about 10% to about 100%, about 10% to about 200%, about 10% to about 300%, about 10% to about 400%, about 10% to about 500%, about 25% to about 50%, about 25% to about 75%, about 25% to about 100%, about 25% to about 200%, about 25% to about 300%, about 25% to about 400%, about 25% to about 500%, about 50% to about 100%, about 50% to about 200%, about 50% to about 300%, about 50% to about 400%, or about 50% to about 500%, relative to naïve CD4⁺ CD25⁻ FoxP3⁻ cells cultured under Treg cell differentiation not exposed to the same RAR antagonist.

In an embodiment, a RAR antagonist has activity that results in increased α4β7 expression in cells exposed to the RAR antagonist. In aspects of this embodiment, a RAR antagonist increases α4β7 expression in cells by at least 10%, at least 20%, at least 30%, at least 40%, at least 50%, at least 60%, at least 70%, at least 80%, at least 90%, at least 100%, at least 200%, at least 300%, at least 400%, or at least 500%, relative to cells not exposed to the same RAR antagonist. In other aspects of this embodiment, a RAR antagonist increases α4β7 expression in cells by about 10% to about 25%, about 10% to about 50%, about 10% to about 75%, about 10% to about 100%, about 10% to about 200%, about 10% to about 300%, about 10% to about 400%, about 10% to about 500%, about 25% to about 50%, about 25% to about 75%, about 25% to about 100%, about 25% to about 200%, about 25% to about 300%, about 25% to about 400%, about 25% to about 500%, about 50% to about 100%, about 50% to about 200%, about 50% to about 300%, about 50% to about 400%, or about 50% to about 500%, relative to cells not exposed to the same RAR antagonist.

In another aspect of this embodiment, a RAR antagonist has activity that results in increased α4β7 expression in naïve CD4⁺ CD25⁻ FoxP3⁻ cells cultured under Treg cell differentiation conditions. In other aspects of this embodiment, a RAR antagonist increases α4β7 expression in naïve CD4⁺ CD25⁻ FoxP3⁻ cells cultured under Treg cell differentiation by at least 10%, at least 20%, at least 30%, at least 40%, at least 50%, at least 60%, at least 70%, at least 80%, at least 90%, at least 100%, at least 200%, at least 300%, at least 400%, or at least 500%, relative to naïve CD4⁺ CD25⁻ FoxP3⁻ cells cultured under Treg cell differentiation not exposed to the same RAR antagonist. In yet other aspects of this embodiment, a RAR antagonist increases α4β7 expression in naïve CD4⁺ CD25⁻ FoxP3⁻ cells cultured under Treg cell differentiation by about 10% to about 25%, about 10% to about 50%, about 10% to about 75%, about 10% to about 100%, about 10% to about 200%, about 10% to about 300%, about 10% to about 400%, about 10% to about 500%, about 25% to about 50%, about 25% to about 75%, about 25% to about 100%, about 25% to about 200%, about 25% to about 300%, about 25% to about 400%, about 25% to about 500%, about 50% to about 100%, about 50% to about 200%, about 50% to about 300%, about 50% to about 400%, or about 50% to about 500%, relative to naïve CD4⁺ CD25⁻ FoxP3⁻ cells cultured under Treg cell differentiation not exposed to the same RAR antagonist.

Aspects of the present specification provide, in part, a RAR antagonist having activity that inhibits Th17 cell differentiation. In aspects of this embodiment, a RAR antagonist inhibits Th17 cell differentiation by at least 10%, at least 20%, at least 30%, at least 40%, at least 50%, at least 60%, at least 70%, at least 80%, at least 90%, at least 100%, at least 200%, at least 300%, at least 400%, or at least 500%. In other aspects of this embodiment, a RAR antagonist inhibits Th17 cell differentiation by about 10% to about 25%, about 10% to about 50%, about 10% to about 75%, about 10% to about 100%, about 10% to about 200%, about 10% to about 300%, about 10% to about 400%, about 10% to about 500%, about 25% to about 50%, about 25% to about 75%, about 25% to about 100%, about 25% to about 200%, about 25% to about 300%, about 25% to about 400%, about 25% to about 500%, about 50% to about 100%, about 50% to about 200%, about 50% to about 300%, about 50% to about 400%, or about 50% to about 500%.

In an embodiment, a RAR antagonist has activity that results in decreased IL-17A expression in cells exposed to the RAR antagonist. In aspects of this embodiment, a RAR antagonist decreases IL-17A expression in cells by at least 10%, at least 20%, at least 30%, at least 40%, at least 50%, at least 60%, at least 70%, at least 80%, at least 90%, at least 100%, at least 200%, at least 300%, at least 400%, or at least 500%, relative to cells not exposed to the same RAR antagonist. In other aspects of this embodiment, a RAR antagonist decreases IL-17A expression in cells by about 10% to about 25%, about 10% to about 50%, about 10% to about 75%, about 10% to about 100%, about 10% to about 200%, about 10% to about 300%, about 10% to about 400%, about 10% to about 500%, about 25% to about 50%, about 25% to about 75%, about 25% to about 100%, about 25% to about 200%, about 25% to about 300%, about 25% to about 400%, about 25% to about 500%, about 50% to about 100%, about 50% to about 200%, about 50% to about 300%, about 50% to about 400%, or about 50% to about 500%, relative to cells not exposed to the same RAR antagonist.

In another aspect of this embodiment, a RAR antagonist has activity that results in decreased IL-17A expression in naïve CD4⁺ CD25⁻ FoxP3⁻ cells cultured under Th17 cell differentiation conditions. In other aspects of this embodiment, a RAR antagonist decreases IL-17A expression in naïve CD4⁺ CD25⁻ FoxP3⁻ cells cultured under Th17 cell differentiation by at least 10%, at least 20%, at least 30%, at least 40%, at least 50%, at least 60%, at least 70%, at least 80%, at least 90%, at least 100%, at least 200%, at least 300%, at least 400%, or at least 500%, relative to naïve CD4⁺ CD25⁻ FoxP3⁻ cells cultured under Th17 cell differentiation not exposed to the same RAR antagonist. In yet other aspects of this embodiment, a RAR antagonist decreases IL-17A expression in naïve CD4⁺ CD25⁻ FoxP3⁻ cells cultured under Th17 cell differentiation by about 10% to about 25%, about 10% to about 50%, about 10% to about 75%, about 10% to about 100%, about 10% to about 200%, about 10% to about 300%, about 10% to about 400%, about 10% to about 500%, about 25% to about 50%, about 25% to about 75%, about 25% to about 100%, about 25% to about 200%, about 25% to about 300%, about 25% to about 400%, about 25% to about 500%, about 50% to about 100%, about 50% to about 200%, about 50% to about 300%, about 50% to about 400%, or about 50% to about 500%, relative to naïve CD4⁺ CD25⁻ FoxP3⁻ cells cultured under Th17 cell differentiation not exposed to the same RAR antagonist.

Aspects of the present specification provide, in part, a RAR antagonist having activity that both promotes Treg cell differentiation and inhibits Th17 cell differentiation. In aspects of this embodiment, a RAR antagonist promotes Treg cell differentiation by at least 10%, at least 20%, at least 30%, at least 40%, at least 50%, at least 60%, at least 70%, at least 80%, at least 90%, at least 100%, at least 200%, at least 300%, at least 400%, or at least 500% as well as inhibits Th17 cell differentiation by at least 10%, at least 20%, at least 30%, at least 40%, at least 50%, at least 60%, at least 70%, at least 80%, at least 90%, at least 100%, at least 200%, at least 300%, at least 400%, or at least 500%. In other aspects of this embodiment, a RAR antagonist promotes Treg cell differentiation by about 10% to about 25%, about 10% to about 50%, about 10% to about 75%, about 10% to about 100%, about 10% to about 200%, about 10% to about 300%, about 10% to about 400%, about 10% to about 500%, about 25% to about 50%, about 25% to about 75%, about 25% to about 100%, about 25% to about 200%, about 25% to about 300%, about 25% to about 400%, about 25% to about 500%, about 50% to about 100%, about 50% to about 200%, about 50% to about 300%, about 50% to about 400%, or about 50% to about 500%, as well as inhibits Th17 cell differentiation by about 10% to about 25%, about 10% to about 50%, about 10% to about 75%, about 10% to about 100%, about 10% to about 200%, about 10% to about 300%, about 10% to about 400%, about 10% to about 500%, about 25% to about 50%, about 25% to about 75%, about 25% to about 100%, about 25% to about 200%, about 25% to about 300%, about 25% to about 400%, about 25% to about 500%, about 50% to about 100%, about 50% to about 200%, about 50% to about 300%, about 50% to about 400%, or about 50% to about 500%.

In an embodiment, a RAR antagonist has activity that results in increased FoxP3 and/or α4β7 expression as well as decreases IL-17A expression in cells exposed to the RAR antagonist. In aspects of this embodiment, a RAR antagonist increases Foxp3 and/or α4β7 expression in cells by at least 10%, at least 20%, at least 30%, at least 40%, at least 50%, at least 60%, at least 70%, at least 80%, at least 90%, at least 100%, at least 200%, at least 300%, at least 400%, or at least 500%, as well as decreases IL-17A expression in cells by at least 10%, at least 20%, at least 30%, at least 40%, at least 50%, at least 60%, at least 70%, at least 80%, at least 90%, at least 100%, at least 200%, at least 300%, at least 400%, or at least 500%, relative to cells not exposed to the same RAR antagonist. In other aspects of this embodiment, a RAR antagonist increases Foxp3 and/or α4β7 expression in cells by about 10% to about 25%, about 10% to about 50%, about 10% to about 75%, about 10% to about 100%, about 10% to about 200%, about 10% to about 300%, about 10% to about 400%, about 10% to about 500%, about 25% to about 50%, about 25% to about 75%, about 25% to about 100%, about 25% to about 200%, about 25% to about 300%, about 25% to about 400%, about 25% to about 500%, about 50% to about 100%, about 50% to about 200%, about 50% to about 300%, about 50% to about 400%, or about 50% to about 500%, as well as decreases IL-17A expression in cells by about 10% to about 25%, about 10% to about 50%, about 10% to about 75%, about 10% to about 100%, about 10% to about 200%, about 10% to about 300%, about 10% to about 400%, about 10% to about 500%, about 25% to about 50%, about 25% to about 75%, about 25% to about 100%, about 25% to about 200%, about 25% to about 300%, about 25% to about 400%, about 25% to about 500%, about 50% to about 100%, about 50% to about 200%, about 50% to about 300%, about 50% to about 400%, or about 50% to about 500%, relative to cells not exposed to the same RAR antagonist.

In another aspect of this embodiment, a RAR antagonist has activity that results in increased FoxP3 and/or α4β7 expression in naïve CD4⁺ CD25⁻ FoxP3⁻ cells cultured under Treg cell differentiation conditions as well as decreases IL-17A expression in naïve CD4⁺ CD25⁻ FoxP3⁻ cells cultured under Th17 cell differentiation conditions. In other aspects of this embodiment, a RAR antagonist increases Foxp3 and/or α4β7 expression in naïve CD4⁺ CD25⁻ FoxP3⁻ cells cultured under Treg cell differentiation by at least 10%, at least 20%, at least 30%, at least 40%, at least 50%, at least 60%, at least 70%, at least 80%, at least 90%, at least 100%, at least 200%, at least 300%, at least 400%, or at least 500%, relative to naïve CD4⁺ CD25⁻ FoxP3⁻ cells cultured under Treg cell differentiation not exposed to the same RAR antagonist as well as decreases IL-17A expression in naïve CD4⁺ CD25⁻ FoxP3⁻ cells cultured under Th17 cell differentiation by at least 10%, at least 20%, at least 30%, at least 40%, at least 50%, at least 60%, at least 70%, at least 80%, at least 90%, at least 100%, at least 200%, at least 300%, at least 400%, or at least 500%, relative to naïve CD4⁺ CD25⁻ FoxP3⁻ cells cultured under Th17 cell differentiation not exposed to the same RAR antagonist.

In yet other aspects of this embodiment, a RAR antagonist increases Foxp3 and/or α4β7 expression in naïve CD4⁺ CD25⁻ FoxP3⁻ cells cultured under Treg cell differentiation by about 10% to about 25%, about 10% to about 50%, about 10% to about 75%, about 10% to about 100%, about 10% to about 200%, about 10% to about 300%, about 10% to about 400%, about 10% to about 500%, about 25% to about 50%, about 25% to about 75%, about 25% to about 100%, about 25% to about 200%, about 25% to about 300%, about 25% to about 400%, about 25% to about 500%, about 50% to about 100%, about 50% to about 200%, about 50% to about 300%, about 50% to about 400%, or about 50% to about 500%, relative to naïve CD4⁺ CD25⁻ FoxP3⁻ cells cultured under Treg cell differentiation not exposed to the same RAR antagonist as well as decreases IL-17A expression in naïve CD4⁺ CD25⁻ FoxP3⁻ cells cultured under Th17 cell differentiation by about 10% to about 25%, about 10% to about 50%, about 10% to about 75%, about 10% to about 100%, about 10% to about 200%, about 10% to about 300%, about 10% to about 400%, about 10% to about 500%, about 25% to about 50%, about 25% to about 75%, about 25% to about 100%, about 25% to about 200%, about 25% to about 300%, about 25% to about 400%, about 25% to about 500%, about 50% to about 100%, about 50% to about 200%, about 50% to about 300%, about 50% to about 400%, or about 50% to about 500%, relative to naïve CD4⁺ CD25⁻ FoxP3⁻ cells cultured under Th17 cell differentiation not exposed to the same RAR antagonist.

Aspects of the present specification provide, in part, a composition comprising a RAR antagonist. A RAR antagonist includes the compounds disclosed herein. The compositions disclosed herein may, or may not, comprise any number and combination of compounds disclosed herein. For instance, a composition can comprise, e.g., two or more compounds disclosed herein, three or more compounds disclosed herein, four or more compounds disclosed herein, or five or more compounds disclosed herein.

A compound disclosed herein, or a composition comprising such a compound, is generally administered to an individual as a pharmaceutical composition. Pharmaceutical compositions may be prepared by combining a therapeutically effective amount of at least one compound as disclosed herein, or a pharmaceutically acceptable salt thereof, as an active ingredient, with conventional acceptable pharmaceutical excipients, and by preparation of unit dosage forms suitable for therapeutic use. As used herein, the term “pharmaceutical composition” and refers to a therapeutically effective concentration of an active compound, such as, e.g., any of the compounds disclosed herein. Preferably, the pharmaceutical composition does not produce an adverse, allergic, or other untoward or unwanted reaction when administered to an individual. A pharmaceutical composition disclosed herein is useful for medical and veterinary applications. A pharmaceutical composition may be administered to an individual alone, or in combination with other supplementary active compounds, agents, drugs or hormones. The pharmaceutical compositions may be manufactured using any of a variety of processes, including, without limitation, conventional mixing, dissolving, granulating, dragee-making, levigating, emulsifying, encapsulating, entrapping, and lyophilizing. The pharmaceutical composition can take any of a variety of forms including, without limitation, a sterile solution, suspension, emulsion, lyophilizate, tablet, pill, pellet, capsule, powder, syrup, elixir, or any other dosage form suitable for administration.

A pharmaceutical composition produced using the methods disclosed herein may be a liquid formulation, semi-solid formulation, or a solid formulation. A formulation disclosed herein can be produced in a manner to form one phase, such as, e.g., an oil or a solid. Alternatively, a formulation disclosed herein can be produced in a manner to form two phase, such as, e.g., an emulsion. A pharmaceutical composition disclosed herein intended for such administration may be prepared according to any method known to the art for the manufacture of pharmaceutical compositions.

Liquid formulations suitable for parenteral injection may comprise physiologically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethyleneglycol (PEG), glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

Semi-solid formulations suitable for topical administration include, without limitation, ointments, creams, salves, and gels. In such solid formulations, the active compound may be admixed with at least one inert customary excipient (or carrier) such as, a lipid and/or polyethylene glycol.

Solid formulations suitable for oral administration include capsules, tablets, pills, powders and granules. In such solid formulations, the active compound may be admixed with at least one inert customary excipient (or carrier) such as sodium citrate or dicalcium phosphate or (a) fillers or extenders, as for example, starches, lactose, sucrose, glucose, mannitol and silicic acid, (b) binders, as for example, carboxymethylcellulose, alignates, gelatin, polyvinylpyrrolidone, sucrose and acacia, (c) humectants, as for example, glycerol, (d) disintegrating agents, as for example, agar-agar, calcium carbonate, potato or tapioca starch, alginic acid, certain complex silicates and sodium carbonate, (e) solution retarders, as for example, paraffin, (f) absorption accelerators, as for example, quaternary ammonium compounds, (g) wetting agents, as for example, cetyl alcohol and glycerol monostearate, (h) adsorbents, as for example, kaolin and bentonite, and (i) lubricants, as for example, talc, calcium stearate, magnesium stearate, solid polyethylene glycols, sodium lauryl sulfate or mixtures thereof. In the case of capsules, tablets and pills, the dosage forms may also comprise buffering agents.

In liquid and semi-solid formulations, a concentration of a therapeutic compound disclosed herein typically may be between about 50 mg/mL to about 1,000 mg/mL. In aspects of this embodiment, a therapeutically effective amount of a therapeutic compound disclosed herein may be from, e.g., about 50 mg/mL to about 100 mg/mL, about 50 mg/mL to about 200 mg/mL, about 50 mg/mL to about 300 mg/mL, about 50 mg/mL to about 400 mg/mL, about 50 mg/mL to about 500 mg/mL, about 50 mg/mL to about 600 mg/mL, about 50 mg/mL to about 700 mg/mL, about 50 mg/mL to about 800 mg/mL, about 50 mg/mL to about 900 mg/mL, about 50 mg/mL to about 1,000 mg/mL, about 100 mg/mL to about 200 mg/mL, about 100 mg/mL to about 300 mg/mL, about 100 mg/mL to about 400 mg/mL, about 100 mg/mL to about 500 mg/mL, about 100 mg/mL to about 600 mg/mL, about 100 mg/mL to about 700 mg/mL, about 100 mg/mL to about 800 mg/mL, about 100 mg/mL to about 900 mg/mL, about 100 mg/mL to about 1,000 mg/mL, about 200 mg/mL to about 300 mg/mL, about 200 mg/mL to about 400 mg/mL, about 200 mg/mL to about 500 mg/mL, about 200 mg/mL to about 600 mg/mL, about 200 mg/mL to about 700 mg/mL, about 200 mg/mL to about 800 mg/mL, about 200 mg/mL to about 900 mg/mL, about 200 mg/mL to about 1,000 mg/mL, about 300 mg/mL to about 400 mg/mL, about 300 mg/mL to about 500 mg/mL, about 300 mg/mL to about 600 mg/mL, about 300 mg/mL to about 700 mg/mL, about 300 mg/mL to about 800 mg/mL, about 300 mg/mL to about 900 mg/mL, about 300 mg/mL to about 1,000 mg/mL, about 400 mg/mL to about 500 mg/mL, about 400 mg/mL to about 600 mg/mL, about 400 mg/mL to about 700 mg/mL, about 400 mg/mL to about 800 mg/mL, about 400 mg/mL to about 900 mg/mL, about 400 mg/mL to about 1,000 mg/mL, about 500 mg/mL to about 600 mg/mL, about 500 mg/mL to about 700 mg/mL, about 500 mg/mL to about 800 mg/mL, about 500 mg/mL to about 900 mg/mL, about 500 mg/mL to about 1,000 mg/mL, about 600 mg/mL to about 700 mg/mL, about 600 mg/mL to about 800 mg/mL, about 600 mg/mL to about 900 mg/mL, or about 600 mg/mL to about 1,000 mg/mL.

In semi-solid and solid formulations, an amount of a therapeutic compound disclosed herein typically may be between about 0.01% to about 45% by weight. In aspects of this embodiment, an amount of a therapeutic compound disclosed herein may be from, e.g., about 0.1% to about 45% by weight, about 0.1% to about 40% by weight, about 0.1% to about 35% by weight, about 0.1% to about 30% by weight, about 0.1% to about 25% by weight, about 0.1% to about 20% by weight, about 0.1% to about 15% by weight, about 0.1% to about 10% by weight, about 0.1% to about 5% by weight, about 1% to about 45% by weight, about 1% to about 40% by weight, about 1% to about 35% by weight, about 1% to about 30% by weight, about 1% to about 25% by weight, about 1% to about 20% by weight, about 1% to about 15% by weight, about 1% to about 10% by weight, about 1% to about 5% by weight, about 5% to about 45% by weight, about 5% to about 40% by weight, about 5% to about 35% by weight, about 5% to about 30% by weight, about 5% to about 25% by weight, about 5% to about 20% by weight, about 5% to about 15% by weight, about 5% to about 10% by weight, about 10% to about 45% by weight, about 10% to about 40% by weight, about 10% to about 35% by weight, about 10% to about 30% by weight, about 10% to about 25% by weight, about 10% to about 20% by weight, about 10% to about 15% by weight, about 15% to about 45% by weight, about 15% to about 40% by weight, about 15% to about 35% by weight, about 15% to about 30% by weight, about 15% to about 25% by weight, about 15% to about 20% by weight, about 20% to about 45% by weight, about 20% to about 40% by weight, about 20% to about 35% by weight, about 20% to about 30% by weight, about 20% to about 25% by weight, about 25% to about 45% by weight, about 25% to about 40% by weight, about 25% to about 35% by weight, or about 25% to about 30% by weight.

A pharmaceutical composition disclosed herein can optionally include a pharmaceutically acceptable carrier that facilitates processing of an active compound into pharmaceutically acceptable compositions. As used herein, the term “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. As used herein, the term “pharmacologically acceptable carrier” is synonymous with “pharmacological carrier” and refers to any carrier that has substantially no long term or permanent detrimental effect when administered and encompasses terms such as “pharmacologically acceptable vehicle, stabilizer, diluent, additive, auxiliary, or excipient.” Such a carrier generally is mixed with an active compound or permitted to dilute or enclose the active compound and can be a solid, semi-solid, or liquid agent. It is understood that the active compounds can be soluble or can be delivered as a suspension in the desired carrier or diluent. Any of a variety of pharmaceutically acceptable carriers can be used including, without limitation, aqueous media such as, e.g., water, saline, glycine, hyaluronic acid and the like; solid carriers such as, e.g., starch, magnesium stearate, mannitol, sodium saccharin, talcum, cellulose, glucose, sucrose, lactose, trehalose, magnesium carbonate, and the like; solvents; dispersion media; coatings; antibacterial and antifungal agents; isotonic and absorption delaying agents; or any other inactive ingredient. Selection of a pharmacologically acceptable carrier can depend on the mode of administration. Except insofar as any pharmacologically acceptable carrier is incompatible with the active compound, its use in pharmaceutically acceptable compositions is contemplated. Non-limiting examples of specific uses of such pharmaceutical carriers can be found in Pharmaceutical Dosage Forms and Drug Delivery Systems (Howard C. Ansel et al., eds., Lippincott Williams & Wilkins Publishers, 7^{th} ed. 1999); Remington: The Science and Practice of Pharmacy (Alfonso R. Gennaro ed., Lippincott, Williams & Wilkins, 20^{th} ed. 2000); Goodman & Gilman's The Pharmacological Basis of Therapeutics (Joel G. Hardman et al., eds., McGraw-Hill Professional, 10^{th} ed. 2001); and Handbook of Pharmaceutical Excipients (Raymond C. Rowe et al., APhA Publications, 4^{th} edition 2003). These protocols are routine and any modifications are well within the scope of one skilled in the art and from the teaching herein.

A pharmaceutical composition disclosed herein can optionally include, without limitation, other pharmaceutically acceptable components (or pharmaceutical components), including, without limitation, buffers, preservatives, tonicity adjusters, salts, antioxidants, osmolality adjusting agents, physiological substances, pharmacological substances, bulking agents, emulsifying agents, wetting agents, sweetening or flavoring agents, and the like. Various buffers and means for adjusting pH can be used to prepare a pharmaceutical composition disclosed herein, provided that the resulting preparation is pharmaceutically acceptable. Such buffers include, without limitation, acetate buffers, borate buffers, citrate buffers, phosphate buffers, neutral buffered saline, and phosphate buffered saline. It is understood that acids or bases can be used to adjust the pH of a composition as needed. Pharmaceutically acceptable antioxidants include, without limitation, sodium metabisulfite, sodium thiosulfate, acetylcysteine, butylated hydroxyanisole, and butylated hydroxytoluene. Useful preservatives include, without limitation, benzalkonium chloride, chlorobutanol, thimerosal, phenylmercuric acetate, phenylmercuric nitrate, a stabilized oxy chloro composition, such as, e.g., sodium chlorite and chelants, such as, e.g., DTPA or DTPA-bisamide, calcium DTPA, and CaNaDTPA-bisamide. Tonicity adjustors useful in a pharmaceutical composition include, without limitation, salts such as, e.g., sodium chloride, potassium chloride, mannitol or glycerin and other pharmaceutically acceptable tonicity adjustor. The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. It is understood that these and other substances known in the art of pharmacology can be included in a pharmaceutical composition useful in the invention.

A compound disclosed herein, or a composition comprising such a compound, may also be incorporated into a drug delivery platform in order to achieve a controlled compound release profile over time. Such a drug delivery platform comprises a compound disclosed herein dispersed within a polymer matrix, typically a biodegradable, bioerodible, and/or bioresorbable polymer matrix. As used herein, the term “polymer” refers to synthetic homo- or copolymers, naturally occurring homo- or copolymers, as well as synthetic modifications or derivatives thereof having a linear, branched or star structure. Copolymers can be arranged in any form, such as, e.g., random, block, segmented, tapered blocks, graft, or triblock. Polymers are generally condensation polymers. Polymers can be further modified to enhance their mechanical or degradation properties by introducing cross-linking agents or changing the hydrophobicity of the side residues. If crosslinked, polymers are usually less than 5% crosslinked, usually less than 1% crosslinked.

Suitable polymers include, without limitation, alginates, aliphatic polyesters, polyalkylene oxalates, polyamides, polyamidoesters, polyanhydrides, polycarbonates, polyesters, polyethylene glycol, polyhydroxyaliphatic carboxylic acids, polyorthoesters, polyoxaesters, polypeptides, polyphosphazenes, polysaccharides, and polyurethanes. The polymer usually comprises at least about 10% (w/w), at least about 20% (w/w), at least about 30% (w/w), at least about 40% (w/w), at least about 50% (w/w), at least about 60% (w/w), at least about 70% (w/w), at least about 80% (w/w), or at least about 90% (w/w) of the drug delivery platform. Examples of biodegradable, bioerodible, and/or bioresorbable polymers and methods useful to make a drug delivery platform are described in, e.g., Drost, et. al., Controlled Release Formulation, U.S. Pat. No. 4,756,911; Smith, et. al., Sustained Release Drug Delivery Devices, U.S. Pat. No. 5,378,475; Wong and Kochinke, Formulation for Controlled Release of Drugs by Combining Hyrophilic and Hydrophobic Agents, U.S. Pat. No. 7,048,946; Hughes, et. al., Compositions and Methods for Localized Therapy of the Eye, U.S. Patent Publication 2005/0181017; Hughes, Hypotensive Lipid-Containing Biodegradable Intraocular Implants and Related Methods, U.S. Patent Publication 2005/0244464; Altman, et al., Silk Fibroin Hydrogels and Uses Thereof, U.S. Patent Publication 2011/0008437; each of which is incorporated by reference in its entirety.

In aspects of this embodiment, a polymer composing the matrix is a polypeptide such as, e.g., silk fibroin, keratin, or collagen. In other aspects of this embodiment, a polymer composing the matrix is a polysaccharide such as, e.g., cellulose, agarose, elastin, chitosan, chitin, or a glycosaminoglycan like chondroitin sulfate, dermatan sulfate, keratan sulfate, or hyaluronic acid. In yet other aspects of this embodiment, a polymer composing the matrix is a polyester such as, e.g., D-lactic acid, L-lactic acid, racemic lactic acid, glycolic acid, caprolactone, and combinations thereof.

One of ordinary skill in the art appreciates that the selection of a suitable polymer for forming a suitable disclosed drug delivery platform depends on several factors. The more relevant factors in the selection of the appropriate polymer(s), include, without limitation, compatibility of polymer with drug, desired release kinetics of drug, desired biodegradation kinetics of platform at implantation site, desired bioerodible kinetics of platform at implantation site, desired bioresorbable kinetics of platform at implantation site, in vivo mechanical performance of platform, processing temperatures, biocompatibility of platform, and patient tolerance. Other relevant factors that, to some extent, dictate the in vitro and in vivo behavior of the polymer include the chemical composition, spatial distribution of the constituents, the molecular weight of the polymer and the degree of crystallinity.

A drug delivery platform includes both a sustained release drug delivery platform and an extended release drug delivery platform. As used herein, the term “sustained release” refers to the release of a compound disclosed herein over a period of about seven days or more. As used herein, the term “extended release” refers to the release of a compound disclosed herein over a period of time of less than about seven days.

In aspects of this embodiment, a sustained release drug delivery platform releases a compound disclosed herein with substantially first order release kinetics over a period of, e.g., about 7 days after administration, about 15 days after administration, about 30 days after administration, about 45 days after administration, about 60 days after administration, about 75 days after administration, or about 90 days after administration. In other aspects of this embodiment, a sustained release drug delivery platform releases a compound disclosed herein with substantially first order release kinetics over a period of, e.g., at least 7 days after administration, at least 15 days after administration, at least 30 days after administration, at least 45 days after administration, at least 60 days after administration, at least 75 days after administration, or at least 90 days after administration.

In aspects of this embodiment, a drug delivery platform releases a compound disclosed herein with substantially first order release kinetics over a period of, e.g., about 1 day after administration, about 2 days after administration, about 3 days after administration, about 4 days after administration, about 5 days after administration, or about 6 days after administration. In other aspects of this embodiment, a drug delivery platform releases a compound disclosed herein with substantially first order release kinetics over a period of, e.g., at most 1 day after administration, at most 2 days after administration, at most 3 days after administration, at most 4 days after administration, at most 5 days after administration, or at most 6 days after administration.

Aspects of the present specification provide, in part, a transplant rejection. Transplant rejection occurs when a transplanted organ or tissue is not accepted by the body of the transplant recipient because the immune system of the recipient attacks the transplanted organ or tissue. An adaptive immune response, transplant rejection is mediated through both T cell-mediated and humoral immune (antibodies) mechanisms. The number of mismatched alleles between the major histocompatibility complex (MHC) antigens on the donor cells, organ, or tissue and the host cells determines the speed and magnitude of the rejection response. Different mechanisms tend to act against different transplants.

A transplant rejection can be classified as a hyperacute rejection, an acute rejection, or a chronic rejection. Hyperacute rejection is a complement-mediated response in recipients with pre-existing antibodies to the donor (for example, ABO blood type antibodies). Hyperacute rejection occurs within minutes after the transplant and the transplant must be immediately removed to prevent a severe systemic inflammatory response. Rapid agglutination of the blood occurs.

Acute rejection may begin as early as one week after transplantation (as opposed to hyperacute rejection, which is immediate). The risk of acute rejection is highest in the first three months after transplantation. However, acute rejection can also occur months to years after transplantation. The reason that acute rejection usually begins one week after transplantation is that T cells are involved in the rejection mechanism. These T cells must differentiate before rejection begins. The T cells cause cells in the transplanted tissue to lyse, or produce cytokines that cause necrosis of the transplanted tissue. A single episode of acute rejection is not a cause for concern if recognized and treated promptly, and rarely leads to organ failure. Acute rejection occurs to some degree in all transplants (except those between identical twins) unless the immune response in altered through the use of immunosuppressive drugs. It is caused by mismatched HLA, which are present on all cells of the body. There are a large number of different alleles of each HLA, so a perfect match between all HLA in the donor tissue and the recipient's body is extremely rare.

Chronic rejection of a transplanted organ or tissue is where the rejection is due to a poorly understood chronic inflammatory and immune response against the transplanted tissue. Chronic rejection after lung transplantation is the leading cause of long-term morbidity and mortality in lung transplant patients

Also included in the term “transplant rejection” is a graft-versus-host disease (GVHD). GVHD is a common complication of allogeneic bone marrow transplantation in which functional immune cells in the transplanted marrow recognize the recipient as “foreign” and mount an immunologic attack. It can also take place in a blood transfusion under certain circumstances. GVHD is divided into acute and chronic forms. The acute or fulminant form of the disease (aGVHD) is normally observed within the first 100 days post-transplant, and is a major challenge to transplants due to associated morbidity and mortality. The chronic form of graft-versus-host-disease (cGVHD) normally occurs after 100 days. The appearance of moderate to severe cases of cGVHD adversely influences long-term survival of both the transplant and the recipient. Acute and chronic GVHD appear to involve different immune cell subsets, different cytokine profiles, somewhat different host targets, and respond differently to treatment.

Acute GVHD is characterized by selective damage to the liver, skin, mucosa, gastrointestinal tract (GI), immune system (the hematopoietic system, e.g., the bone marrow and the thymus) itself, and the lungs in the form of idiopathic pneumonitis. Acute GVHD of the GI tract can result in severe intestinal inflammation, sloughing of the mucosal membrane, severe diarrhea, abdominal pain, nausea, and vomiting. This is typically diagnosed via intestinal biopsy. Liver GVHD is measured by the bilirubin level in acute patients. Skin GVHD results in a diffuse maculopapular rash, sometimes in a lacy pattern. Acute GVHD is staged as follows: overall grade (skin-liver-gut) with each organ staged individually from a low of 1 (I) to a high of 4 (IV). Patients with grade IV GVHD usually have a poor prognosis. If the GVHD is severe and requires intense immunosuppression involving steroids and additional agents to get under control, the patient may develop severe infections as a result of the immunosuppression and may die of infection. Chronic GVHD also attacks the above organs, but over its long-term course can also cause damage to the connective tissue and exocrine glands.

Aspects of the present disclosure provide methods of treating GVHD and methods of preventing GVHD by administration of an RAR antagonist, such as a RARα or RARγ antagonist. Other aspects of the disclosure provide methods of reducing a symptom associated with a transplant rejection or a GVHD disorder. Non-limiting examples of a symptom reduced by a method of treating a transplant rejection or a GVHD disorder disclosed herein include rash, raised, or discolored areas, thickening or tightening of the skin, abdominal swelling, yellow discoloration of the skin and/or eyes, dry eyes or vision changes, dry mouth, white patches inside the mouth, pain or sensitivity to spicy foods, shortness of breath or changes seen on an chest X-ray, difficulty swallowing, pain with swallowing, weight loss, fatigue, muscle weakness, or pain, increased urinary frequency, burning or bleeding with urination, vaginal dryness or tightening, or penile dysfunction. The methods disclosed herein reduce one or more, two or more, three or more, four or more, five or more, six or more, seven or more, or eight or more symptoms of GVHD.

Aspects of the present disclosure provide, in part, a mammal. A mammal includes a human, and a human can be a patient. Other aspects of the present invention provide, in part, an individual. An individual includes a mammal and a human, and a human can be a patient.

Aspects of the present disclosure provide, in part, administering a compound or a composition disclosed herein. As used herein, the term “administering” means any delivery mechanism that provides a compound or a composition disclosed herein to an individual that potentially results in a clinically, therapeutically, or experimentally beneficial result.

Administration of a compound or a composition disclosed herein include a variety of enteral or parenteral approaches including, without limitation, oral administration in any acceptable form, such as, e.g., tablet, liquid, capsule, powder, or the like; topical administration in any acceptable form, such as, e.g., drops, spray, creams, gels or ointments; buccal, nasal, and/or inhalation administration in any acceptable form; rectal administration in any acceptable form; vaginal administration in any acceptable form; intravascular administration in any acceptable form, such as, e.g., intravenous bolus injection, intravenous infusion, intra-arterial bolus injection, intra-arterial infusion and catheter instillation into the vasculature; pen- and intra-tissue administration in any acceptable form, such as, e.g., intraperitoneal injection, intramuscular injection, subcutaneous injection, subcutaneous infusion, intraocular injection, retinal injection, or sub-retinal injection or epidural injection; intravesicular administration in any acceptable form, such as, e.g., catheter instillation; and by placement device, such as, e.g., an implant, a stent, a patch, a pellet, a catheter, an osmotic pump, a suppository, a bioerodible delivery system, a non-bioerodible delivery system or another implanted extended or slow release system. An exemplary list of biodegradable polymers and methods of use are described in, e.g., Handbook of Biodegradable Polymers (Abraham J. Domb et al., eds., Overseas Publishers Association, 1997).

A compound or a composition disclosed herein can be administered to a mammal using a variety of routes. Routes of administration suitable for treating a transplant rejection or a GVHD disorder as disclosed herein include both local and systemic administration. Local administration results in significantly more delivery of a composition to a specific location as compared to the entire body of the mammal, whereas, systemic administration results in delivery of a composition to essentially the entire body of the individual. Routes of administration suitable for or treating a transplant rejection or a GVHD disorder as disclosed herein also include both central and peripheral administration. Central administration results in delivery of a compound or a composition to essentially the central nervous system of the individual and includes, e.g., intrathecal administration, epidural administration as well as a cranial injection or implant. Peripheral administration results in delivery of a compound or a composition to essentially any area of an individual outside of the central nervous system and encompasses any route of administration other than direct administration to the spine or brain. The actual route of administration of a compound or a composition disclosed herein used can be determined by a person of ordinary skill in the art by taking into account factors, including, without limitation, the type of a transplant rejection or a GVHD disorder, the severity of the GVHD or transplant rejection, the duration of treatment desired, the degree of relief desired, the duration of relief desired, the particular compound or composition used, the rate of excretion of the compound or composition used, the pharmacodynamics of the compound or composition used, the nature of the other compounds to be included in the composition, the particular route of administration, the particular characteristics, history and risk factors of the individual, such as, e.g., age, weight, general health and the like, the response of the individual to the treatment, or any combination thereof. An effective dosage amount of a compound or a composition disclosed herein can thus readily be determined by the person of ordinary skill in the art considering all criteria and utilizing his best judgment on the individual's behalf.

Aspects of the present specification provide, in part, administering a therapeutically effective amount of a compound or a composition disclosed herein. As used herein, the term “therapeutically effective amount” is synonymous with “therapeutically effective dose” and when used in reference to treating a transplant rejection or a GVHD disorder means the minimum dose of a compound or composition disclosed herein necessary to achieve the desired therapeutic effect and includes a dose sufficient to treat the transplant rejection or GVHD and/or reduce at least one symptom associated with a transplant rejection or a GVHD disorder. In aspects of this embodiment, a therapeutically effective amount of a compound or a composition disclosed herein reduces a symptom associated with a transplant rejection or a GVHD disorder by, e.g., at least 10%, at least 20%, at least 30%, at least 40%, at least 50%, at least 60%, at least 70%, at least 80%, at least 90% or at least 100%. In other aspects of this embodiment, a therapeutically effective amount of a compound or a composition disclosed herein reduces a symptom associated with a transplant rejection or a GVHD disorder by, e.g., at most 10%, at most 20%, at most 30%, at most 40%, at most 50%, at most 60%, at most 70%, at most 80%, at most 90% or at most 100%. In yet other aspects of this embodiment, a therapeutically effective amount of a compound or a composition disclosed herein reduces a symptom associated with a transplant rejection or a GVHD disorder by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%. In still other aspects of this embodiment, a therapeutically effective amount of a compound or a composition disclosed herein is the dosage sufficient to reduces a symptom associated with a transplant rejection or a GVHD disorder for, e.g., at least one week, at least one month, at least two months, at least three months, at least four months, at least five months, at least six months, at least seven months, at least eight months, at least nine months, at least ten months, at least eleven months, or at least twelve months.

The amount of active component in a compound or a composition disclosed herein for treating a transplant rejection or a GVHD disorder can be varied so that a suitable dosage is obtained. The actual therapeutically effective amount of a compound or a composition disclosed herein to be administered to a mammal can be determined by a person of ordinary skill in the art by taking into account factors, including, without limitation, the type of the GVHD or transplant rejection, the severity of the GVHD or transplant rejection, the duration of treatment desired, the degree of relief desired, the duration of relief desired, the particular compound or composition used, the rate of excretion of the compound or composition used, the pharmacodynamics of the compound or composition used, the nature of the other compounds to be included in the composition, the particular route of administration, the particular characteristics, history and risk factors of the individual, such as, e.g., age, weight, general health and the like, the response of the individual to the treatment, or any combination thereof. An effective dosage amount of a compound or a composition disclosed herein can thus readily be determined by the person of ordinary skill in the art considering all criteria and utilizing his best judgment on the individual's behalf.

Additionally, where repeated administration of a compound or a composition disclosed herein is used, the actual effective amount of a compound or a composition disclosed herein will further depend upon factors, including, without limitation, the frequency of administration, the half-life of the compound or composition disclosed herein, or any combination thereof. In is known by a person of ordinary skill in the art that an effective amount of a compound or a composition disclosed herein can be extrapolated from in vitro assays and in vivo administration studies using animal models prior to administration to humans. Wide variations in the necessary effective amount are to be expected in view of the differing efficiencies of the various routes of administration. For instance, oral administration generally would be expected to require higher dosage levels than administration by intravenous or intravitreal injection. Variations in these dosage levels can be adjusted using standard empirical routines of optimization, which are well-known to a person of ordinary skill in the art. The precise therapeutically effective dosage levels and patterns are preferably determined by the attending physician in consideration of the above-identified factors.

As a non-limiting example, when administering a compound or a composition disclosed herein to a mammal, a therapeutically effective amount generally is in the range of about 0.001 mg/kg/day to about 100.0 mg/kg/day. In aspects of this embodiment, an effective amount of a compound or a composition disclosed herein can be, e.g., about 0.01 mg/kg/day to about 0.1 mg/kg/day, about 0.03 mg/kg/day to about 3.0 mg/kg/day, about 0.1 mg/kg/day to about 3.0 mg/kg/day, or about 0.3 mg/kg/day to about 3.0 mg/kg/day. In yet other aspects of this embodiment, a therapeutically effective amount of a compound or a composition disclosed herein can be, e.g., at least 0.001 mg/kg/day, at least 0.01 mg/kg/day, at least 0.1 mg/kg/day, at least 1.0 mg/kg/day, at least 10 mg/kg/day, or at least 100 mg/kg/day. In yet other aspects of this embodiment, a therapeutically effective amount of a compound or a composition disclosed herein can be, e.g., at most 0.001 mg/kg/day, at most 0.01 mg/kg/day, at most 0.1 mg/kg/day, at most 1.0 mg/kg/day, at most 10 mg/kg/day, or at most 100 mg/kg/day.

As another non-limiting example, when administering a compound or a composition disclosed herein to a mammal, a therapeutically effective amount generally is in the range of about 0.001 mg/m²/day to about 100.0 mg/m²/day. In aspects of this embodiment, an effective amount of a compound or a composition disclosed herein can be, e.g., about 0.01 mg/m²/day to about 0.1 mg/m²/day, about 0.03 mg/m²/day to about 3.0 mg/m²/day, about 0.1 mg/m²/day to about 3.0 mg/m²/day, or about 0.3 mg/m²/day to about 3.0 mg/m²/day. In yet other aspects of this embodiment, a therapeutically effective amount of a compound or a composition disclosed herein can be, e.g., at least 0.001 mg/m²/day, at least 0.01 mg/m²/day, at least 0.1 mg/m²/day, at least 1.0 mg/m²/day, at least 10 mg/m²/day, or at least 100 mg/m²/day. In yet other aspects of this embodiment, a therapeutically effective amount of a compound or a composition disclosed herein can be, e.g., at most 0.001 mg/m²/day, at most 0.01 mg/m²/day, at most 0.1 mg/m²/day, at most 1.0 mg/m²/day, at most 10 mg/m²/day, or at most 100 mg/m²/day.

Dosing can be single dosage or cumulative (serial dosing), and can be readily determined by one skilled in the art. For instance, treatment of a transplant rejection or a GVHD disorder may comprise a one-time administration of an effective dose of a compound or a composition disclosed herein. As a non-limiting example, an effective dose of a compound or a composition disclosed herein can be administered once to a mammal, e.g., as a single injection or deposition at or near the site exhibiting a symptom of a transplant rejection or a GVHD disorder or a single oral administration of the compound or a composition. Alternatively, treatment of a transplant rejection or a GVHD disorder may comprise multiple administrations of an effective dose of a compound or a composition disclosed herein carried out over a range of time periods, such as, e.g., daily, once every few days, weekly, monthly or yearly. As a non-limiting example, a compound or a composition disclosed herein can be administered once or twice weekly to a mammal. The timing of administration can vary from mammal to mammal, depending upon such factors as the severity of a mammal's symptoms. For example, an effective dose of a compound or a composition disclosed herein can be administered to a mammal once a month for an indefinite period of time, or until the mammal no longer requires therapy. A person of ordinary skill in the art will recognize that the condition of the mammal can be monitored throughout the course of treatment and that the effective amount of a compound or a composition disclosed herein that is administered can be adjusted accordingly.

A compound or a composition disclosed herein as disclosed herein can also be administered to a mammal in combination with other therapeutic compounds to increase the overall therapeutic effect of the treatment. The use of multiple compounds to treat an indication can increase the beneficial effects while reducing the presence of side effects. Exemplary therapeutic compounds include, but are not limited to, cytotoxic compounds, antibacterial compounds, immune modulating compounds, antiviral compounds, immune suppressive compounds, antifungal compounds, analgesic compounds, etc.

### EXAMPLES

The following non-limiting examples are provided for illustrative purposes only in order to facilitate a more complete understanding of representative embodiments now contemplated. These examples should not be construed to limit any of the embodiments described in the present specification, including those pertaining to the methods of treating a transplant rejection or a GVHD disorder, using the RAR antagonists disclosed herein, uses of a RAR antagonist, disclosed herein to manufacture a medicament and/or treat a transplant rejection or a GVHD disorder, methods of promoting Treg cell differentiation in an individual, inhibiting Th17 cell differentiation, or both, as well as uses of a RAR antagonist disclosed herein to promote Treg cell differentiation in an individual, inhibit Th17 cell differentiation, or both.

### Example 1

### Vitamin A Metabolism is Up-Regulated During GVHD

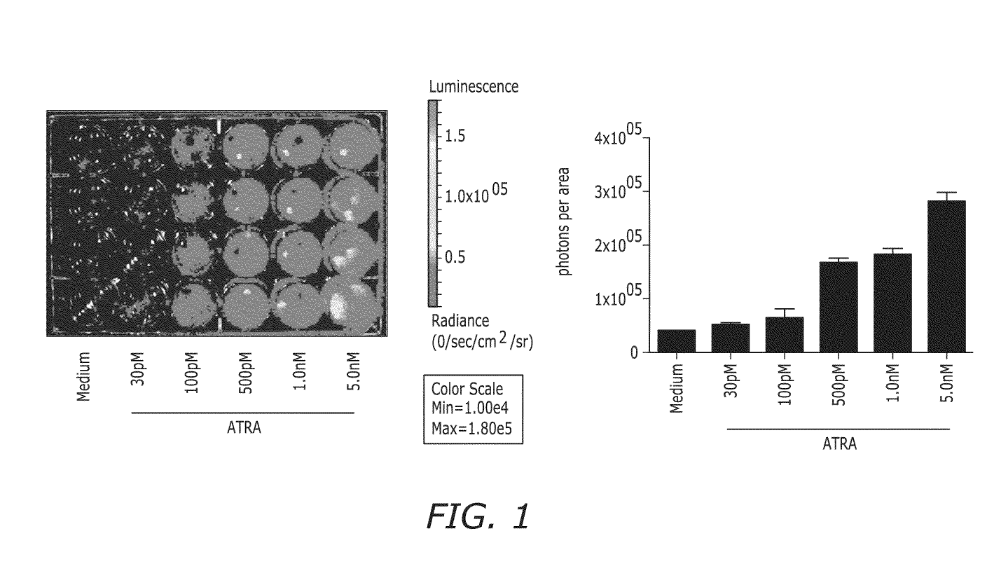

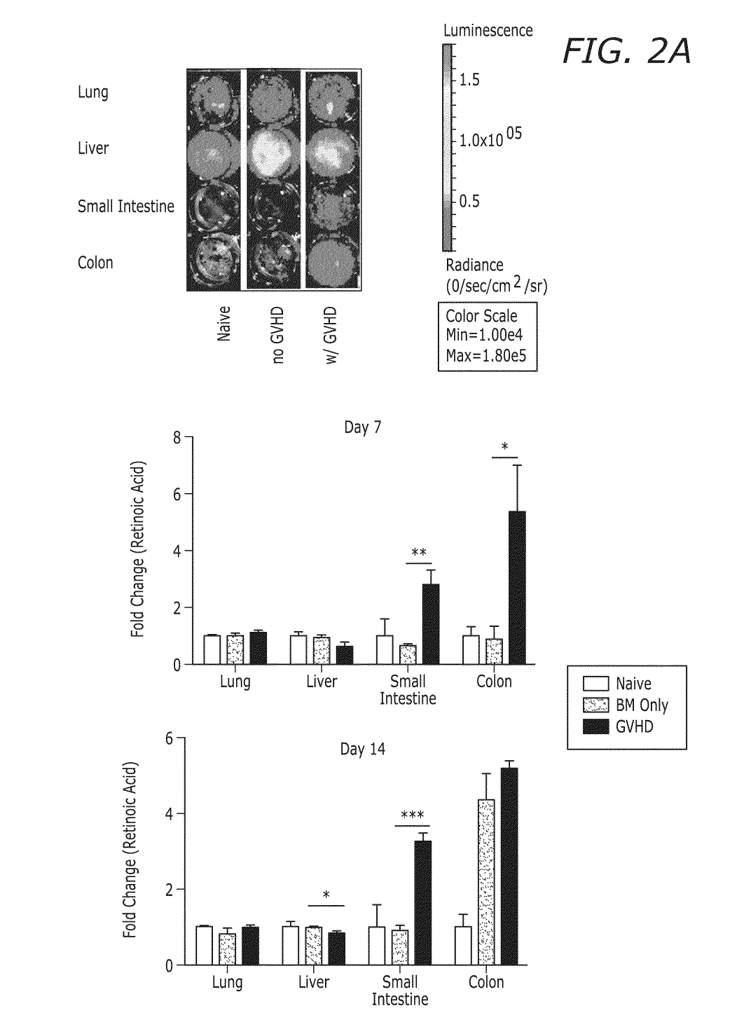

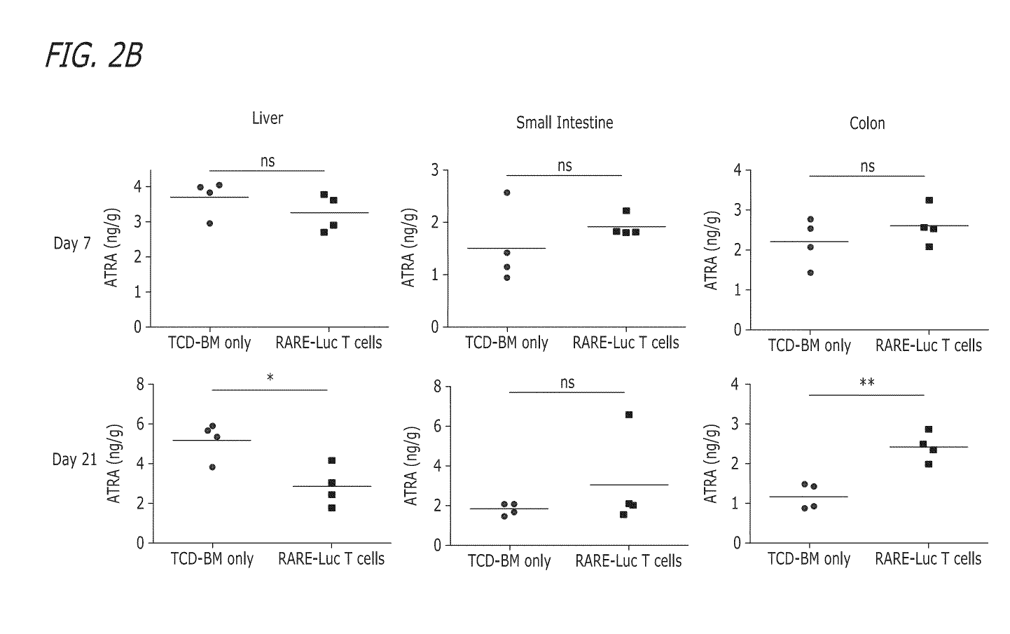

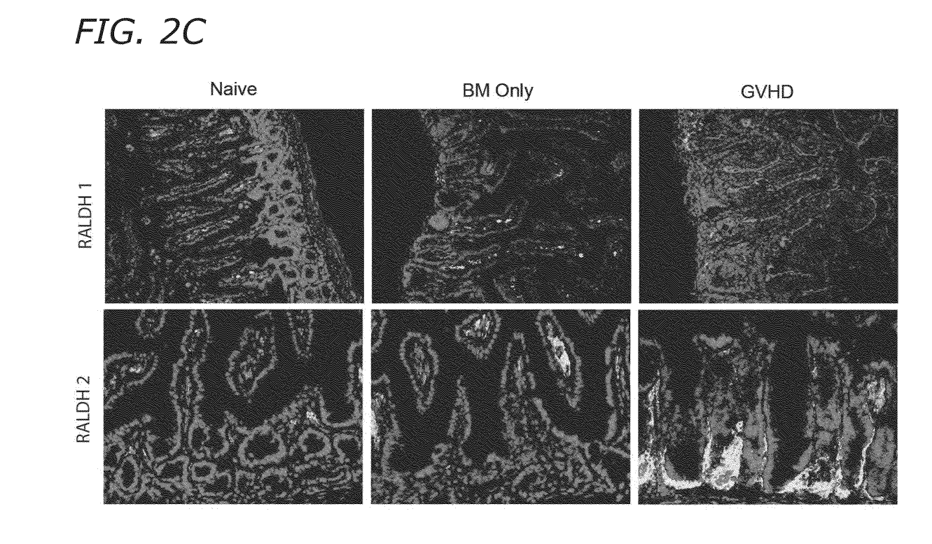

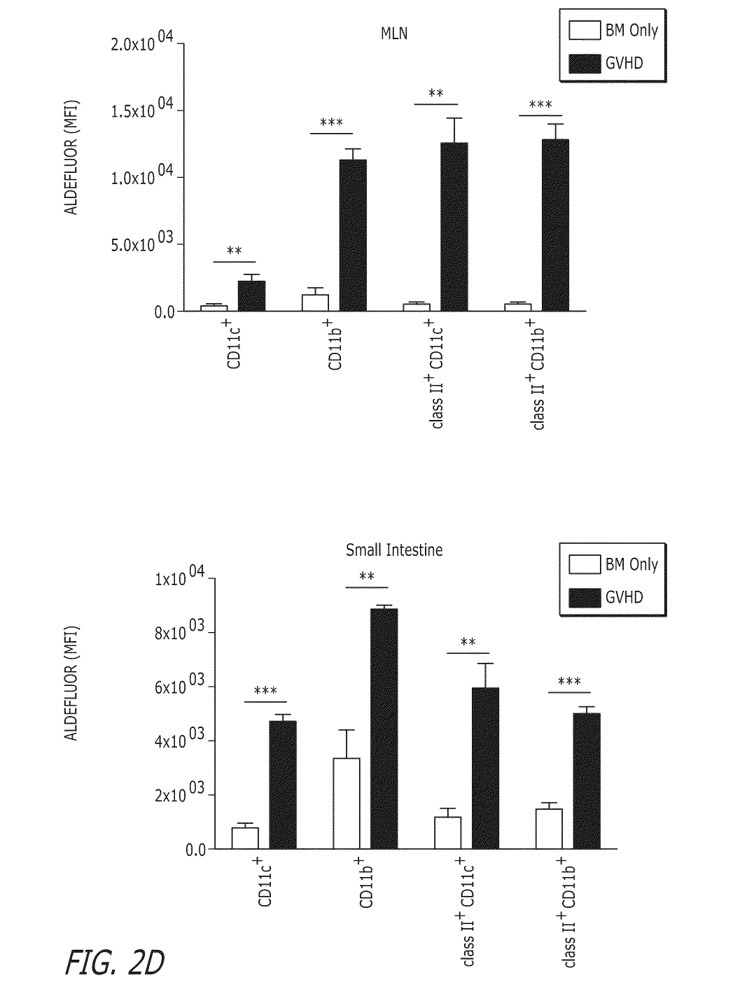

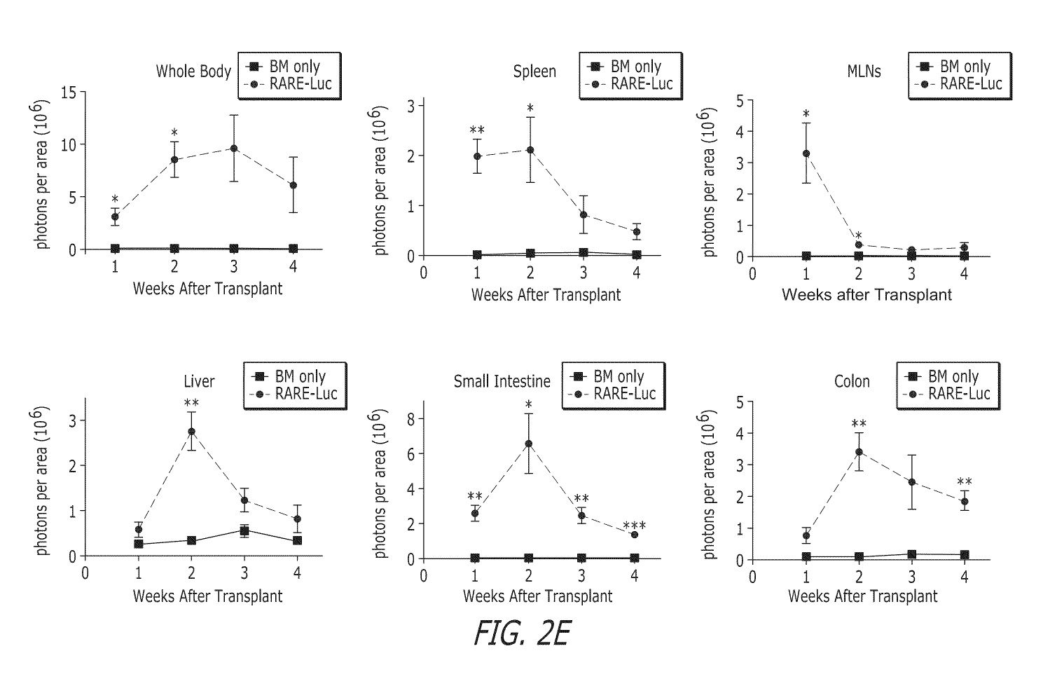

RA levels were quantified in GVHD organs using a B16 tumor line modified to express retinoic acid response element-responsive luciferase (RARE-luc) (B16-DR5 assay) to assess RA signaling as a surrogate assay for RA production. As little as 30 pM of RA can be detected by biolumenescent imaging (BLI) as significantly different than media in this assay (FIG. 1A). For quantification of RA in vivo, lethally irradiated B10.BR mice were given B6 T cell depleted (TCD) BM with or without splenocytes (15×0⁶) to induce GVHD. Lung, liver, small intestine and colon samples were analyzed on days 7 and 14 after BMT (FIG. 2A). RARE-luc signaling induced by small intestine tissue extracts obtained from GVHD mice was significantly higher on days 7 and 14 than that in non-GVHD mice and non-BMT controls. Whereas colon extracts isolated on day 7 from GVHD mice results in high RARE-luc signaling, by day 14 both BMT groups had high and equivalent RARE-luc signaling, suggesting a late radiation effect. In contrast, RARE-luc signaling by day 14 liver extracts was significantly lower in GVHD vs non-GVHD mice (FIG. 2A, p<0.02).

Vitamin A metabolites (ATRA; 13-cis-RA) capable of signaling via RARRXR heterodimer interactions with RAREs were quantified by LC/MS-MS in GVHD organs at weeks 1 and 3 post-BMT. In the liver, ATRA levels were equivalent on day 7 and significantly reduced by day 21 (FIG. 2B, n=4/group), consistent with days 7 and 14 B16-DR5 assay results. Although ATRA levels were not augmented in the small intestine at either time, a minority (25%) of GVHD mice had >3.5-fold higher ATRA levels on day 14 post-BMT and significantly higher day 7 post-BMT 13-cis-RA levels than non-GVHD mice, the latter consistent with B16-DR5 assay results. ATRA levels also were significantly higher in the colon of GVHD vs non-GVHD mice by 3 weeks post-BMT.

Vitamin A is metabolized by alcohol dehydrogenases into retinal, and irreversibly converted to RA by selectively expressed retinal dehydrogenases (RALDHs) in DCs and stromal cells in both Peyer's patches and mesenchymal lymph nodes (MLNs). To determine whether vitamin A metabolizing enzyme expression and functional activity were influenced by GVHD, we first analyzed RALDH1 and RALDH2 expression by immunohistochemistry in small intestinal epithelial cells and RALDH2 in small intestinal DCs in BMT mice on day 14 post-BMT. FIG. 2C shows GVHD induced both RALDH1 and RALDH2 expression in small intestinal non-hematopoietic cells (epithelial or stromal cells) and RALDH2 in CD11c⁺ cells (dendritic cells, DCs). Aldehyde dehydrogenase (ALDH) enzymatic activity was determined by ALDEFLUOR™ (Stem Cell Technologies) staining. Mean fluorescent intensity (MFI) was significantly higher in MHC class DCs from MLNs and lamina propria lymphocytes (LPL) in mice with GVHD versus no GVHD on day 14 post-BMT (FIG. 2D), indicative of functional vitamin A metabolizing capacity.

To determine how irradiation and GVHD influenced donor T cell RA signaling, we employed a novel B6 transgenic strain of mice, B10.BR, in which RARE is positioned upstream of a luc reporter. Lethally irradiated allogeneic B10.BR mice were given B6 BM with or without 3×10⁶ B6 RARE-luc transgenic T cells. Sequential BLI was performed weekly. Total body BLI signals increased in GVHD mice between weeks 1 and 3 (FIG. 2E). By week 4, signals modestly decreased, suggesting that alloreactive T cells destroyed or inhibited the RA producing capacity of host cells. In support of this hypothesis, RAR signaling in donor T cells in MLNs peaked at week 1 then rapidly declined. Signaling of donor T cell RARE-luc T cells in the colon markedly increased between weeks 1 and 2, consistent with increased donor T cell infiltration and/or increased donor T cell RARE-luc signaling on a per-cell basis. A similar pattern of high RARE-luc donor T cell signaling was observed in sites of high endogenous vitamin A metabolizing enzymes. In sites with lower basal RA levels and non-inflamed skin, BLI signals were low (<10⁶ photons/organ/sec). High photon emissions in the small intestine and colon of GVHD mice are possibly a consequence of donor T cell infiltration early post-BMT and T cell infiltration and high donor T cell RAR signaling at week 3 post-BMT. These results indicate that allogeneic BMT affects vitamin A distribution and metabolism.

### Example 2

### High RA Levels Lead to GVHD Acceleration

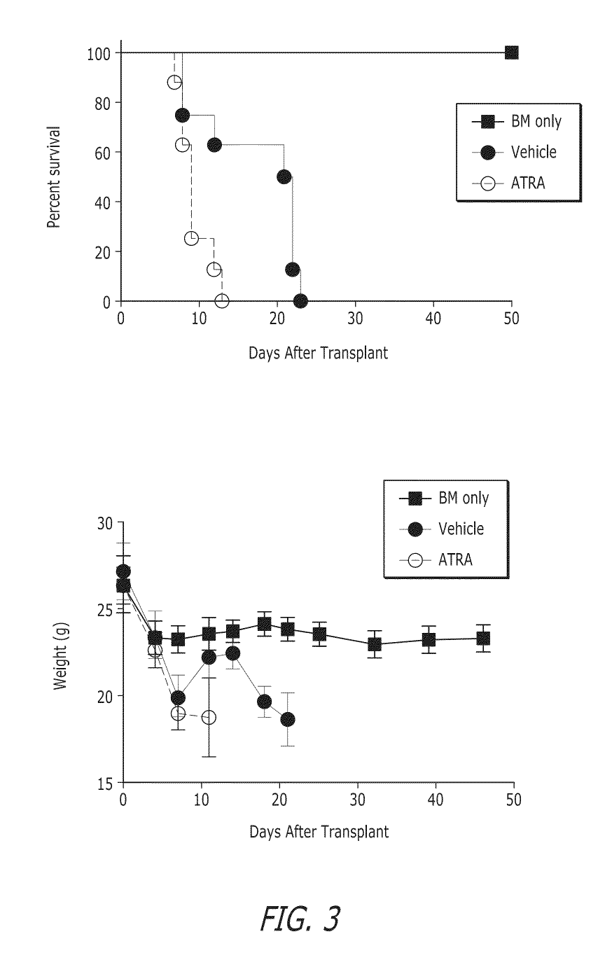

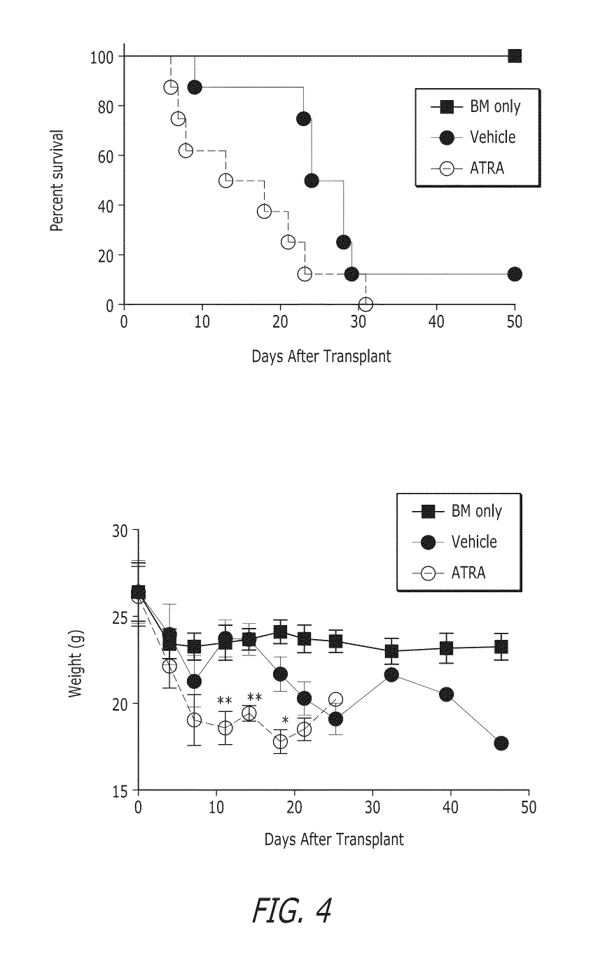

To determine whether heightened vitamin A signaling would augment acute GVHD, B10.BR recipients given BM with or without 15×10⁶ splenocytes from B6 mice were given ATRA (200 μg/mouse IP) daily from day 0 to 28. Surprisingly, recipients given ATRA had a significant increase (p=0.013) in GVHD lethality with a median survival time (MST) of only 9 days vs. 21.5 days for vehicle recipients and had accelerated weight loss (FIG. 3). A similar reduction in survival rates (p=0.037) and weight loss was seen with 5×10⁶ splenocytes (FIG. 4). These findings indicate heightened vitamin A levels and RAR signaling can enhance GVHD lethality.

### Example 3

### Inhibiting RARα Signaling in Donor T Cells Prevents GVHD Lethality and Target Organ Injury

Given the acceleration of GVHD in mice treated with ATRA, we hypothesized that blocking RAR signaling in donor T cells would reduce GVHD. Since RARα expression on CD4⁺ and CD8⁺ T cells has been shown by RT-PCR and up-regulated in activated T cells, we investigated whether RARs have unique functions in the murine GVHD model. To circumvent the severe phenotype of RARα null mutant mice, donor mice that express a dominant negative form of RARα, RAR403 (dnRARα) were used to restrict RAR signaling deficiency to T cells. CD4⁺ T cells from dnRARα-CD4^{Cre} vs dnRARα mice have intact in vitro and in vivo alloproliferative responses or the expression of chemokine receptors and activation antigens (CD44, CD62Lo, CD69), while CD8⁺ T cells had intact activation and day 4 antigen-specific clonal expansion.

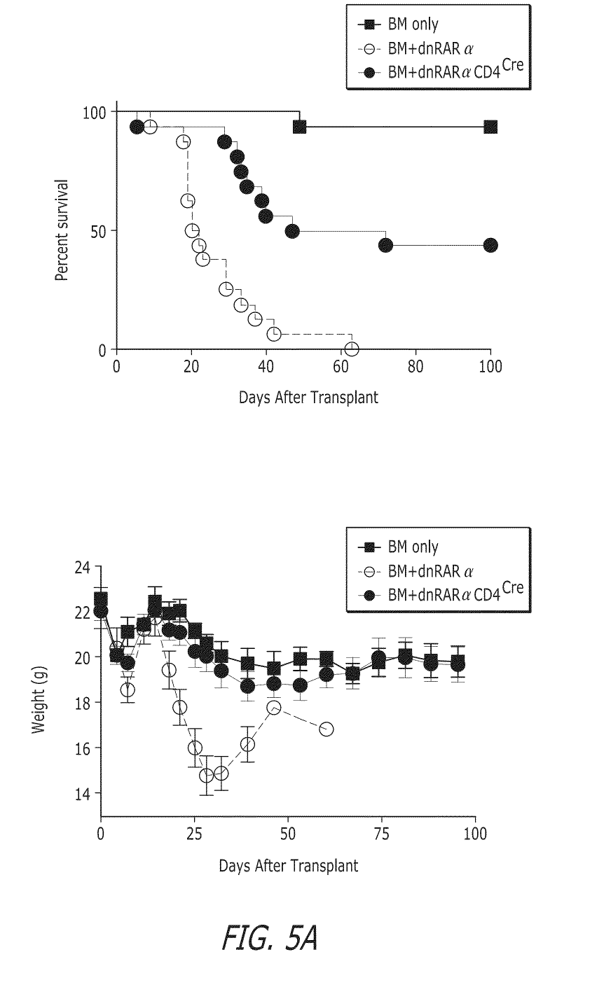

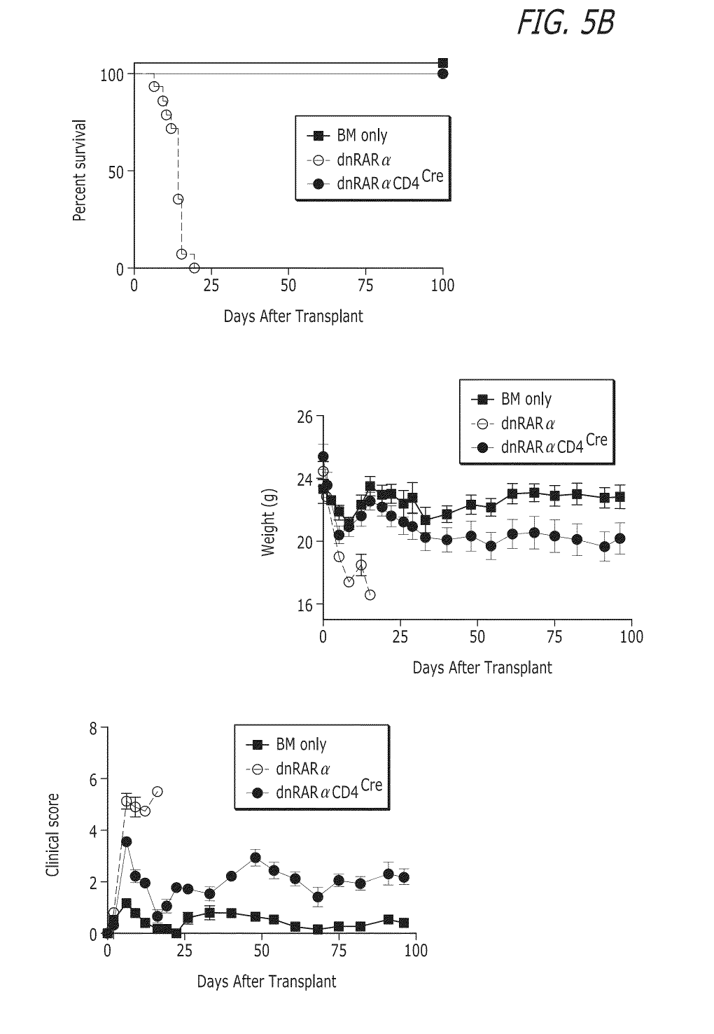

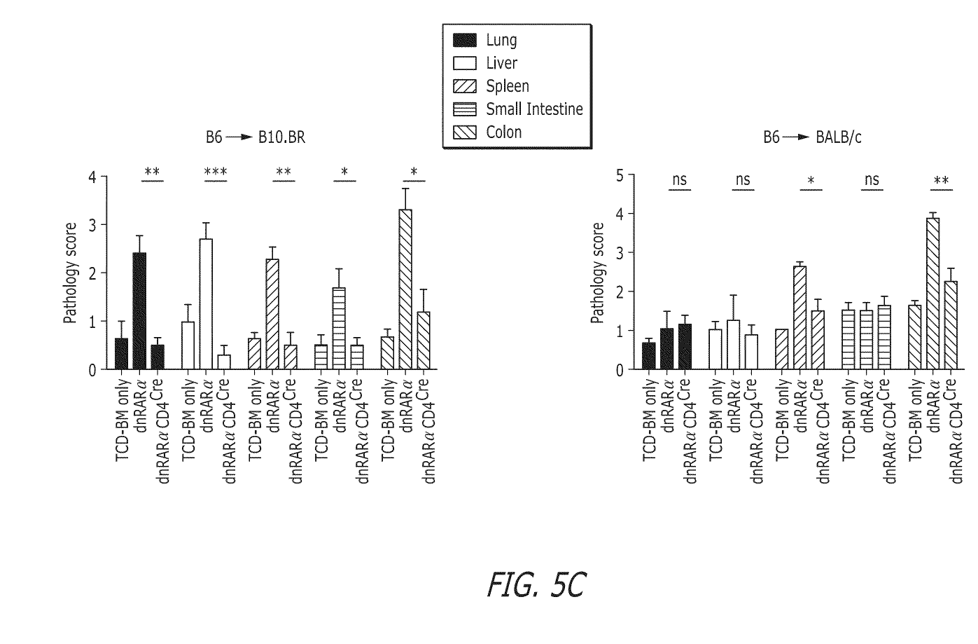

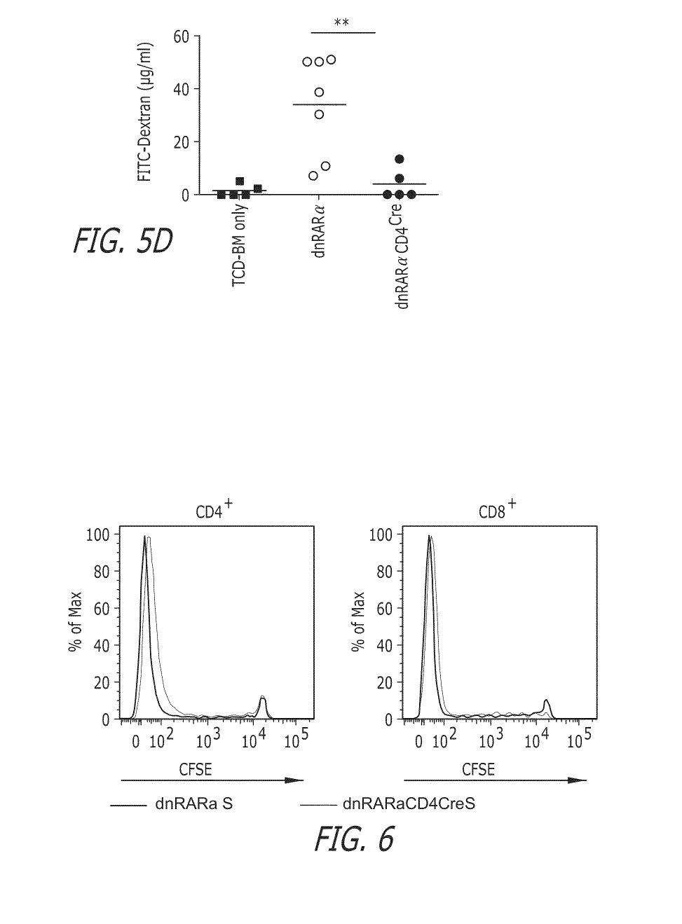

Lethally irradiated B10.BR mice were given B6 BM with or without 10×10⁶ splenocytes from dnRARα-CD4^{Cre} or dnRARα (no CD4^{Cre}, RARα signaling intact) mice as controls. Whereas all controls died from GVHD, 44% mice given dnRARα-CD4^{Cre} survived significantly longer (p<0.001) beyond 100 days post-BMT with mean weight curves similar to BM only controls (FIG. 5A). To determine whether the impact of vitamin A signaling in GVHD was strain dependent, BALB/c recipients were given dnRARαCD4^{Cre} or dnRARα splenocytes (FIG. 5B). Whereas dnRARα splenocytes recipients had rapid lethality and mean weight loss (within 3 weeks post-BMT), none of those receiving dnRARα-CD4^{Cre} died by day 100 (p<0.001) and mean weight curves comparable to BM only controls for the first 1 month post-BMT. Surviving recipients of dnRARα-CD4^{Cre} splenocytes showed significantly lower clinical GVHD scores than BM controls, confirmed by histopathology of the lung, spleen, liver, small intestine, and the colon in B10.BR recipients of B6 cells and the spleen and colon in BALB/c recipients of B6 cells on day 21 (FIG. 5C). Using a FITC-dextran assay to measure intestine epithelial cell barrier function, recipients of dnRARα-CD4^{Cre} splenocytes were found to have reduced intestinal permeability and thus intestinal injury on day 14 post-BMT, compared with those receiving dnRARα splenocytes (FIG. 5D; p=0.007). These results indicate that RAR signaling in donor T cells impacts the mortality/morbidity caused by GVHD.

### Example 4

### Intestinal Homing Receptor Expression and Th1 Phenotypes are Modulated by Inhibiting Donor T Cell RARα Signaling

To elucidate the contributions of RAR signaling inhibition in donor T cells in the development of GVHD, day 4 post-BMT T cell expansion and apoptosis were evaluated in BALB/c recipients using carboxyfluorescein succinimidyl ester (CFSE) labeling and apoptosis by annexin V co-staining. dnRARα-CD4^{Cre} vs dnRARα donor CD4⁺ and CD8⁺ T cells did not have altered early post-BMT proliferation (FIG. 6). Apoptosis frequency (annexin-V⁺) was comparable between groups in CD8⁺ donor T cells, although dnRARα-CD4^{Cre} vs dnRARα CD4⁺ T cells had a reduction in apoptosis frequency (p=0.018, not shown).

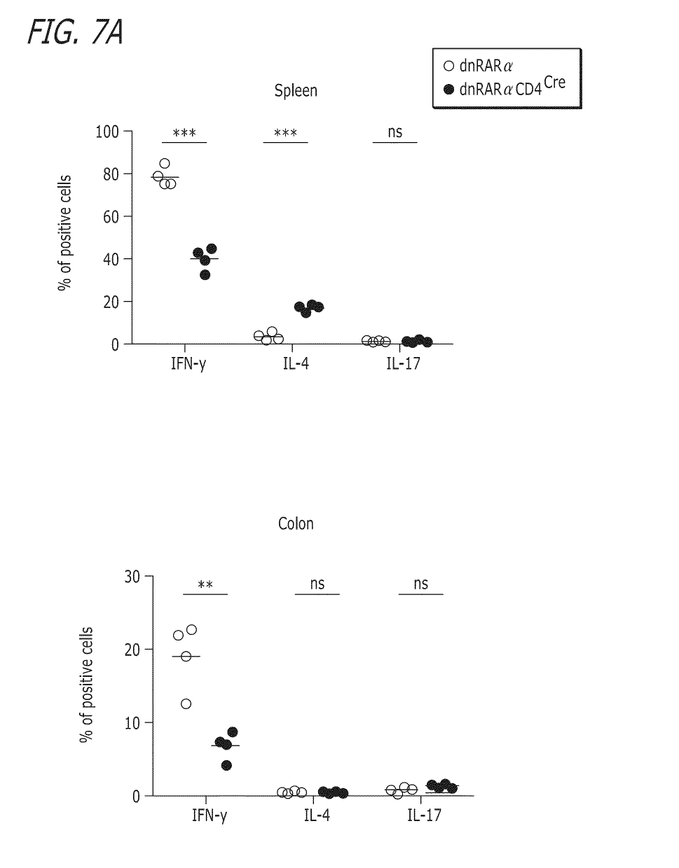

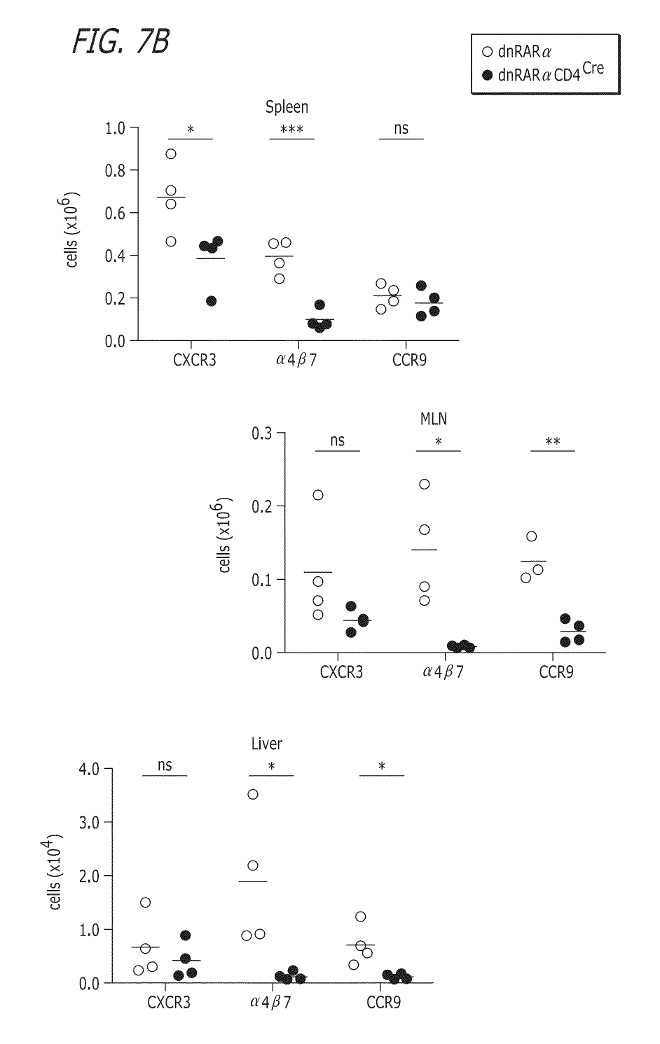

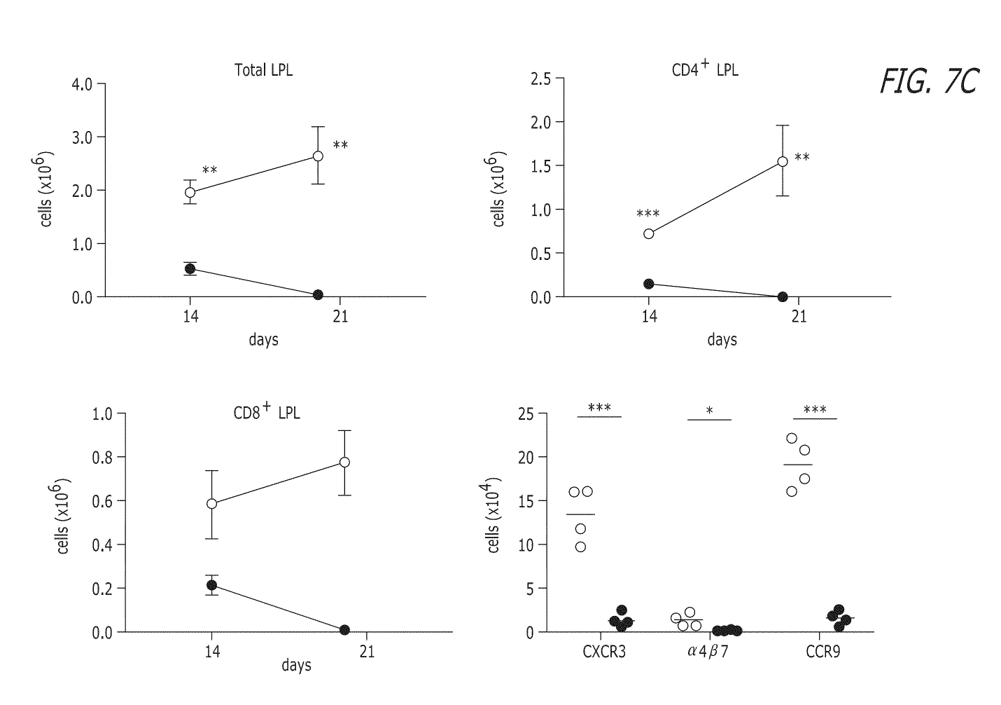

To determine whether RARα signaling affects T helper (Th) generation, splenocytes on day 6 and colonic LPL on day 14 after BMT from BALB/c recipients of dnRARα-CD4^{Cre} or dnRARα T cells were analyzed. IFN-γ-secreting splenic and colon LPL CD4⁺ T cells from dnRARα-CD4^{Cre} vs dnRARα donors were lower in frequency (FIG. 7A), whereas the frequency of splenic IL-4-secreting CD4⁺ T cells was increased (FIG. 7A). Although several reports suggest RAR signaling decreases Th17 generation, there was no significant difference in the frequency of IL-17-secreting CD4⁺ T cells in spleens and colonic LPL during GVHD (FIG. 7A).

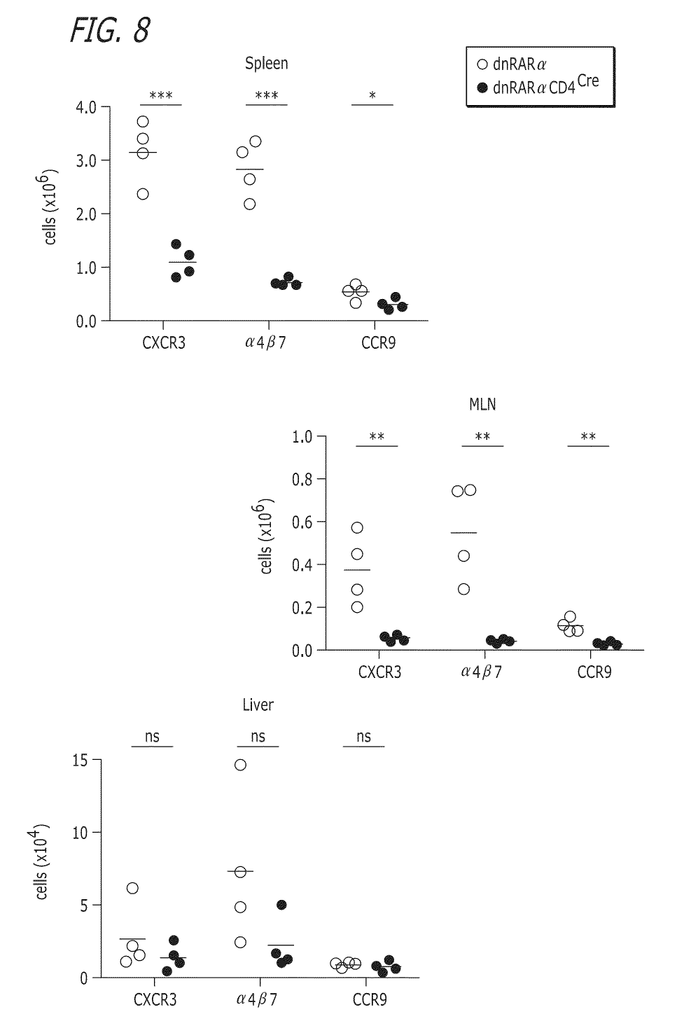

RA has been shown to induce the up-regulation of α4β7 and CCR9 in CD4⁺ and CD8⁺ T cells in vitro in the absence of DCs. Thus, RAR signaling in donor T cells might diminish chemokine receptor and integrin expression that is required for optimal GVHD initiation Thus, we quantified the number and frequency of chemokine and integrin expressing donor CD4⁺ and CD8⁺ T cells post-BMT. Consistent with the improved survival of BALB/c recipients of dnRARα-CD4^{Cre} vs dnRARα donor T cells, on day 6 post-BMT, there were significantly lower numbers of splenic CXCR3 and α4β7⁺ CD4⁺ T cells, MLN and liver α4β7⁺ and CCR9⁺ CD4⁺ (FIG. 7B) as well as splenic and MLN CXCR3⁺, α4β7⁺ and CCR9⁺ CD8⁺ (FIG. 8) T cells. Because CXCR3, α4β7 and CCR9 are important intestinal homing receptors, we also quantified colonic LPL CD4⁺ and CD8⁺ T cells on days 14 and 20 post-BMT. Total, CD4⁺ and CD8⁺ T cells each were significantly reduced in number in recipients of recipients of dnRARα-CD4^{Cre} vs dnRARα T cells (FIG. 7C). Moreover, the numbers of CXCR3, α4β7 and CCR9 expressing LPL were significantly lower in recipients of dnRARα-CD4^{Cre} vs dnRARα T cells. On day 14 post-BMT, the numbers of CXCR3, α4β7 and CCR9 expressing donor T cells were either not significantly reduced or sometimes increased in recipients of dnRARα-CD4^{Cre} vs dnRARα T cells. Thus, dnRARα-CD4^{Cre} vs dnRARα donor T cells have reduced expression of chemokine receptors and integrins early-post-BMT that likely assists their migration into these GVHD sites to initiate the GVHD process.

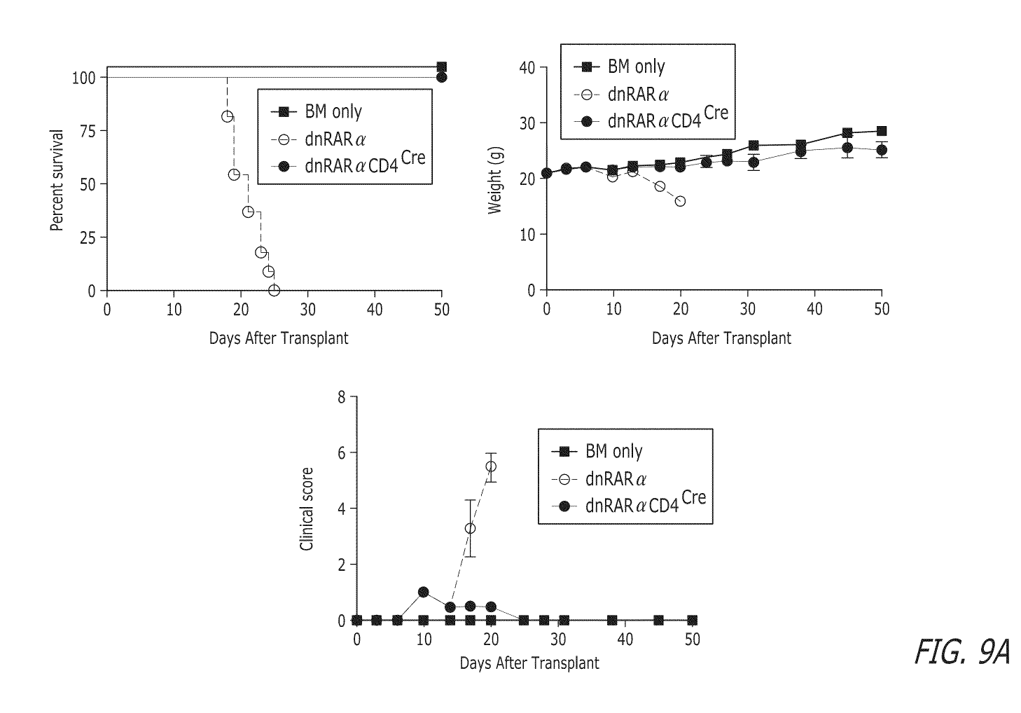

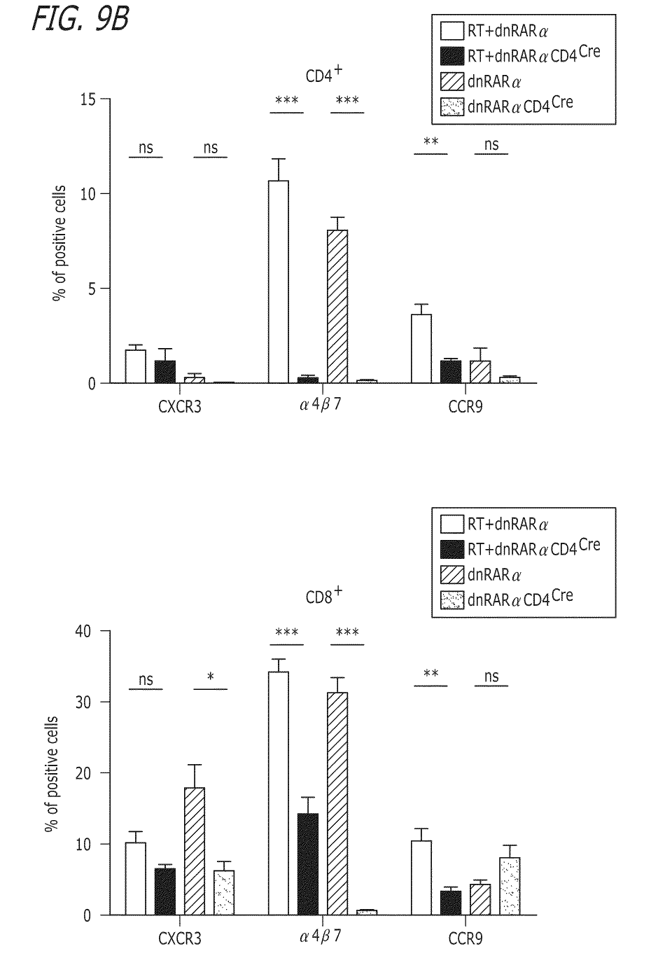

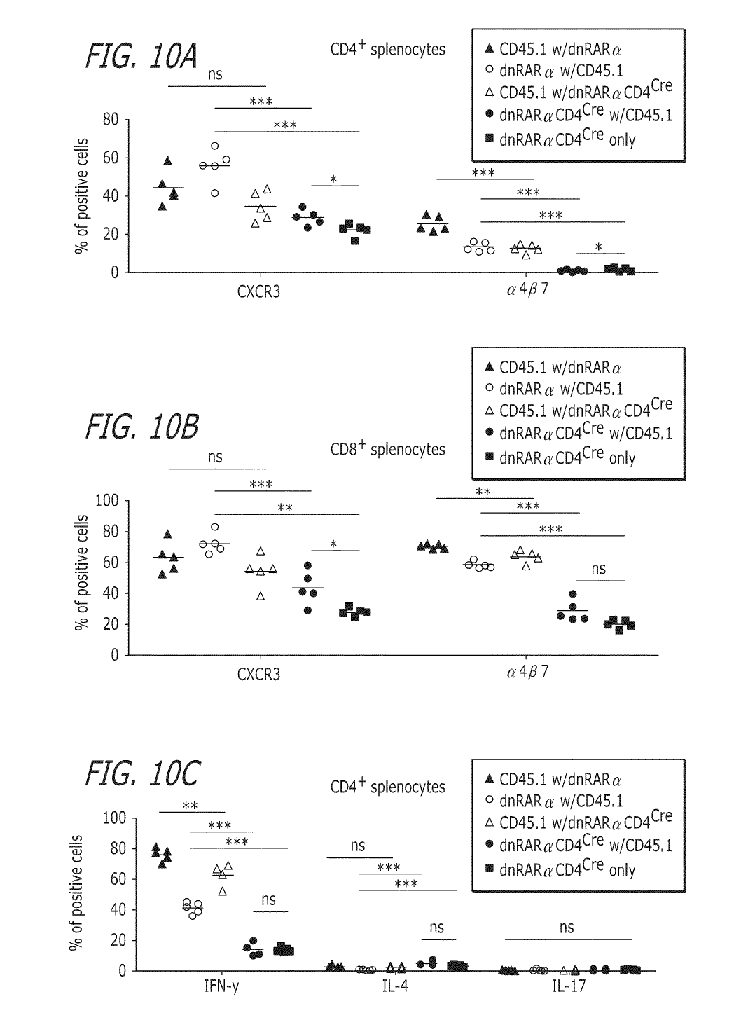

One explanation for the reduced donor T cell migration into GVHD organs seen in recipients of dnRARα-CD4^{Cre} vs dnRARα T cells is that inhibited RAR signaling per se precludes the up-regulation of chemokine receptor and integrin expression needed for appropriate migration into GVHD organs. Alternatively, the reduced production of Teff cytokines we observed might be caused by a RAR signaling defect that prevented the full up-regulation of these receptors and the production of Teff cytokines that contribute to GVHD initiation. To discern between these possibilities, BALB/c recipients were given dnRARα-CD4^{Cre} vs dnRARα T cells along with B6-CD45 congenic (CD45.1⁺) T cells (1:1) to provide a fully intact source of Teff cytokines. An additional group included dnRARα-CD4^{Cre} donor T cells alone (FIG. 9). Frequencies of splenic T cells expressing these receptors originating from dnRARα-CD4^{Cre} vs dnRARα, or congenic T cells were quantified on day 7 post-BMT. CXCR3, but α4β7, was expressed at a significantly higher frequency of CD4+ and CD8+ T cells isolated from the spleen of recipients given dnRARα-CD4Cre with CD45.1+ T cells vs dnRARα-CD4Cre T cells alone. As expected, lower frequencies of CXCR3 and α4β7 CD4+ and CD8+ T cells were seen in the spleen of recipients of dnRARα-CD4Cre vs dnRARα T cells. Whereas, also as expected, a lower frequency of CD4+ IFN-γ+ and higher frequency of CD4+ IL-4+ was found in recipients given dnRARα-CD4Cre vs dnRARα donor T cells, no difference in cytokine activity in donor CD4+ T cells was found between recipients given dnRARαCD4Cre with CD45.1+ T cells vs dnRARα-CD4Cre T cells alone. To further investigate the role of RAR signaling to donor T cell induced GVHD and α4β7 expression in the absence of irradiation conditioning and its associated proinflammatory cytokine release, unirradiated B6D2F1 recipients were given 12×107 splenocytes from syngeneic or allogeneic dnRARα-CD4Cre or dnRARα B6 mice. Recipients of dnRARα splenocytes died within 30 days, whereas all recipients of dnRARα-CD4Cre splenocytes survived beyond 50 days after splenocyte transfer (FIG. 10A). Mean weight curves and clinical scores paralleled the survival curves. Moreover, mice given dnRARα-CD4Cre splenocytes had a significantly lower frequency of α4β7-expressing CD4+ and CD8+ T cells and CXCR3-expressing CD8+ T cells (FIG. 10B). Taken together, these data demonstrate dnRARα-CD4^{Cre} T cells may have defect in α4β7 expression.

### Example 5

### RAR Signaling Inhibition in dnRARα-CD4^{Cre} Donor T Cells Supports Treg Induction and Expansion

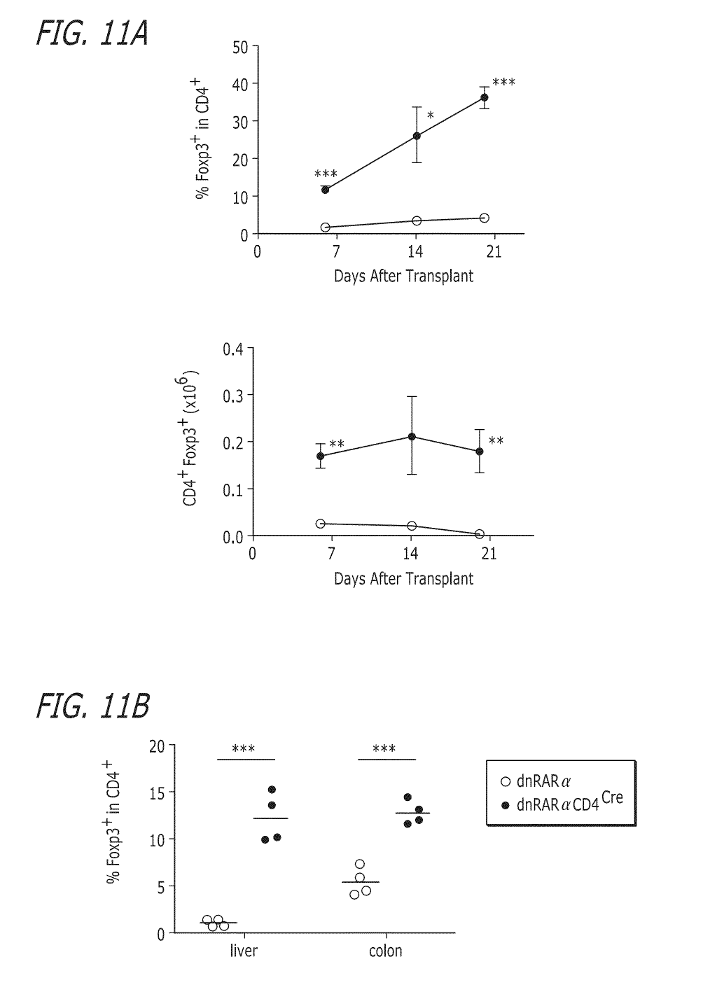

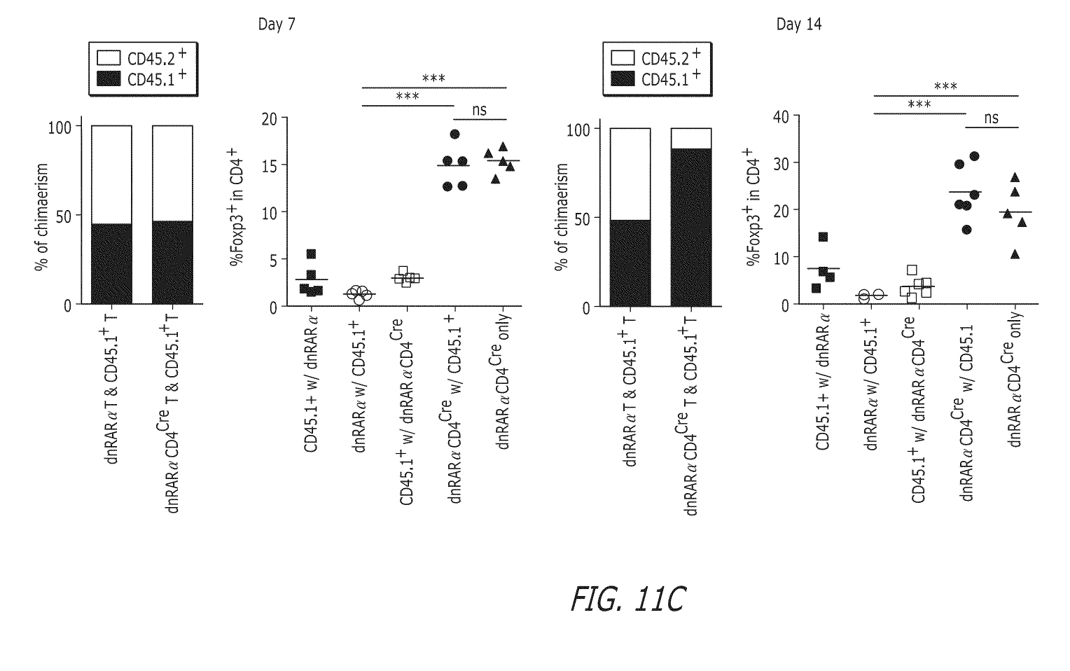

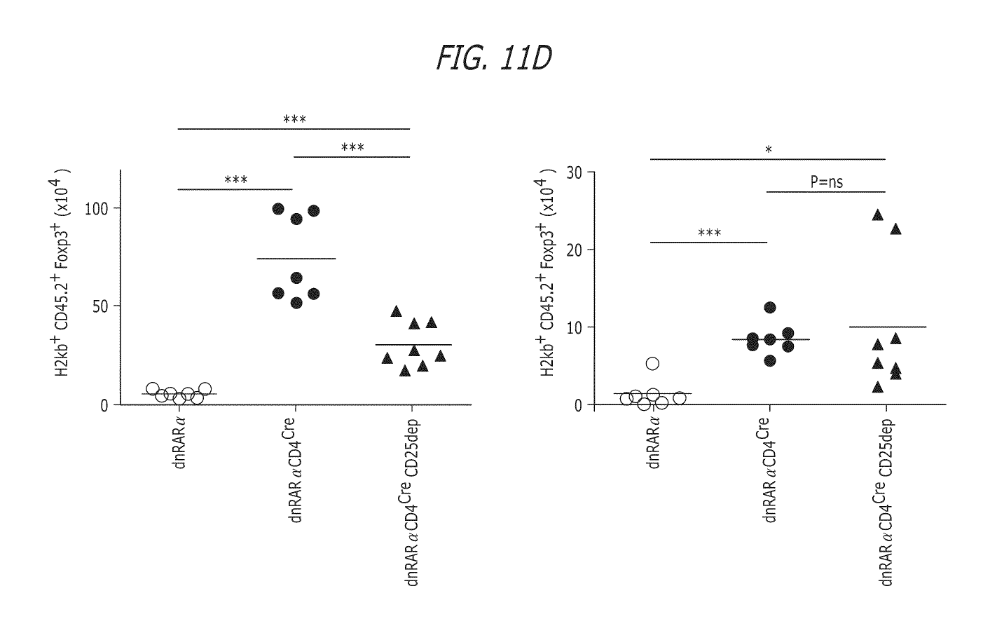

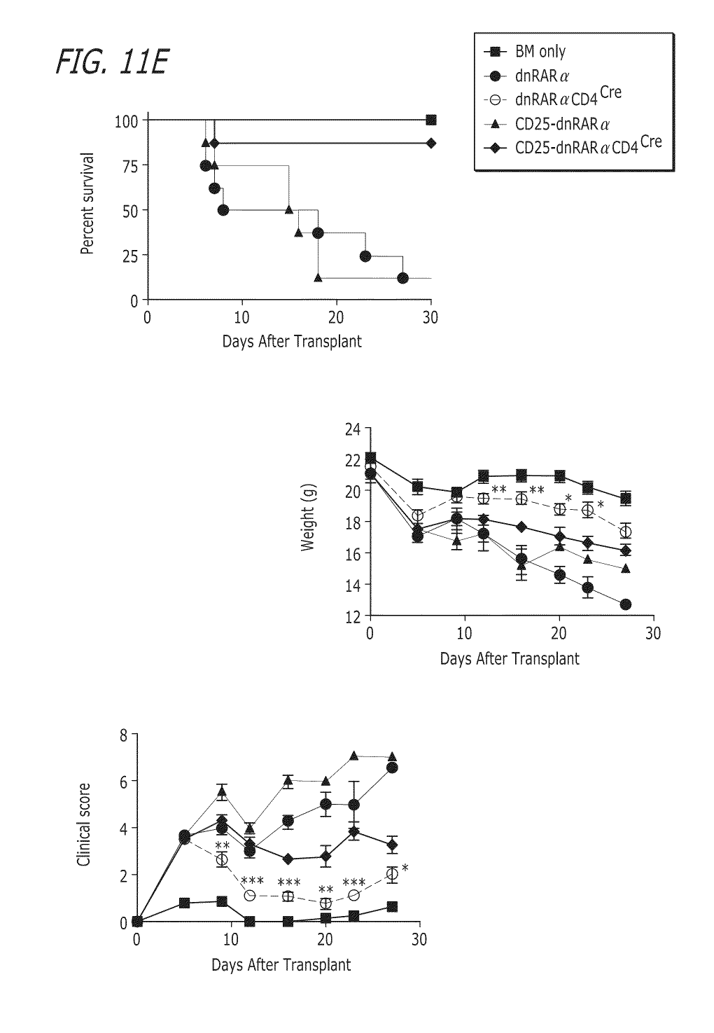

Donor Tregs are potent GVHD suppressors but how RAR signaling affects Treg generation or expansion in vivo during acute GVHD is undetermined. We quantified the frequency and number of CD4⁺ Foxp3⁺ T cells in spleen post-BMT. BALB/c recipients given dnRARα-CD4^{Cre} vs dnRARα T cells had significantly higher frequencies and absolute numbers of splenic Foxp3⁺ cells on days 6, 14 and 20 (FIG. 11A) and a significantly higher frequency of liver and colonic CD4⁺ Foxp3⁺ T cells on day 14 post-BMT (FIG. 11B).

To determine whether donor T cell derived cytokines affect Tregs post-BMT, dnRARα or dnRARα-CD4^{Cre} T cells were co-transferred with CD45.1 T cells (1:1) into BALB/c recipients and the frequency of splenic and MLN donor Tregs was analyzed. On days 7 and 14 post-BMT, a higher frequency of CD4⁺ Foxp3⁺ T cells was seen in recipients of dnRARα-CD4^{Cre} vs dnRARα T cells (with or without co-transferred T cells) (FIG. 11C). Few CD4⁺ Foxp3⁺ T cells were directly derived from CD45.1⁺ or dnRARα T cells, indicating that dnRARα-CD4^{Cre} T cells had a higher propensity to support Tregs, regardless of the presence of co-transferred and CD45.1⁺ T cells. Of note, dnRARαCD4^{Cre} vs dnRARα T cell chimerism was comparable to CD45.1⁺ T cells on day 7; however, dnRARα-CD4^{Cre} vs dnRARα T cells had reduced accumulation by day 14 post-BMT, consistent with a recent report for CD8⁺ T cells.

RA signaling with TGFβ, can support, whereas high proinflammatory cytokine levels can adversely affect, inducible Treg generation. Thus, we sought to determine whether inhibiting RAR signaling would influence donor inducible Treg generation in the context of GVHD. BALB/c mice were given highly Treg-depleted T cells or non-depleted T cells from dnRARα or dnRARα-CD4^{Cre} donors along with B6 CD45.1 congenic BM. On day 14, significantly more Tregs derived from the donor T cell inocula were detected in spleens of recipients of dnRARα-CD4^{Cre} vs dnRARα Treg-replete T cells (FIG. 11D). More Tregs also were seen in recipients of dnRARα-CD4^{Cre} Treg-depleted vs dnRARα Treg-replete T cells, suggesting that dnRARα-CD4^{Cre} donor T cells favored inducible Treg generation. The numbers of CD45.1⁺ Tregs (BM-derived) in the spleens of recipients of dnRARα-CD4^{Cre} Treg-replete T cells and dnRARα-CD4^{Cre} Treg-depleted T cells were comparable and significantly higher than for dnRARα Treg-replete T cells, consistent with enhanced inducible Treg generation from the small numbers of T cells contained in the BM inoculum (FIG. 11D).

To determine whether the GVHD-protective effect of the dnRARα-CD4^{Cre} vs dnRARα donor T cells was dependent upon the presence of donor Tregs, Tregs were depleted from the donor inoculum prior to transfer. Recipients of dnRARα-CD4^{Cre} splenocytes had less severe GVHD than those receiving dnRARα-CD4^{Cre}Treg-depleted splenocytes. Mice in both groups survived significantly longer than recipients of dnRARα splenocytes or dnRARα Treg-depleted splenocytes (FIG. 11E). Mean weights and clinical scores parallel survival curves. Thus, Treg-depletion reduced but did not eliminate the survival benefits of dnRARα-CD4^{Cre} splenocytes.

### Example 6

### GVL Effects of dnRARα-CD4^{Cre} Donor T Cells

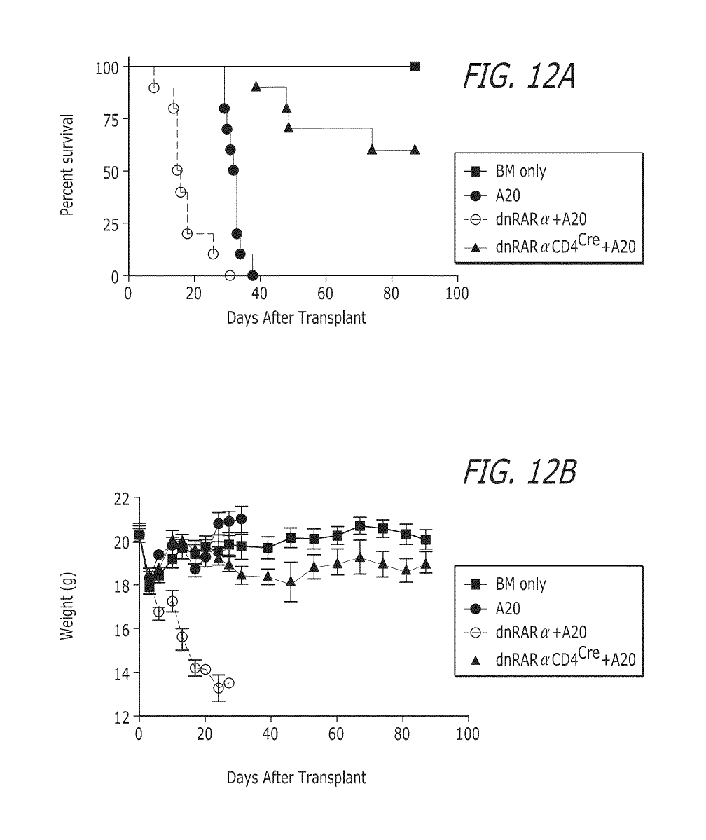

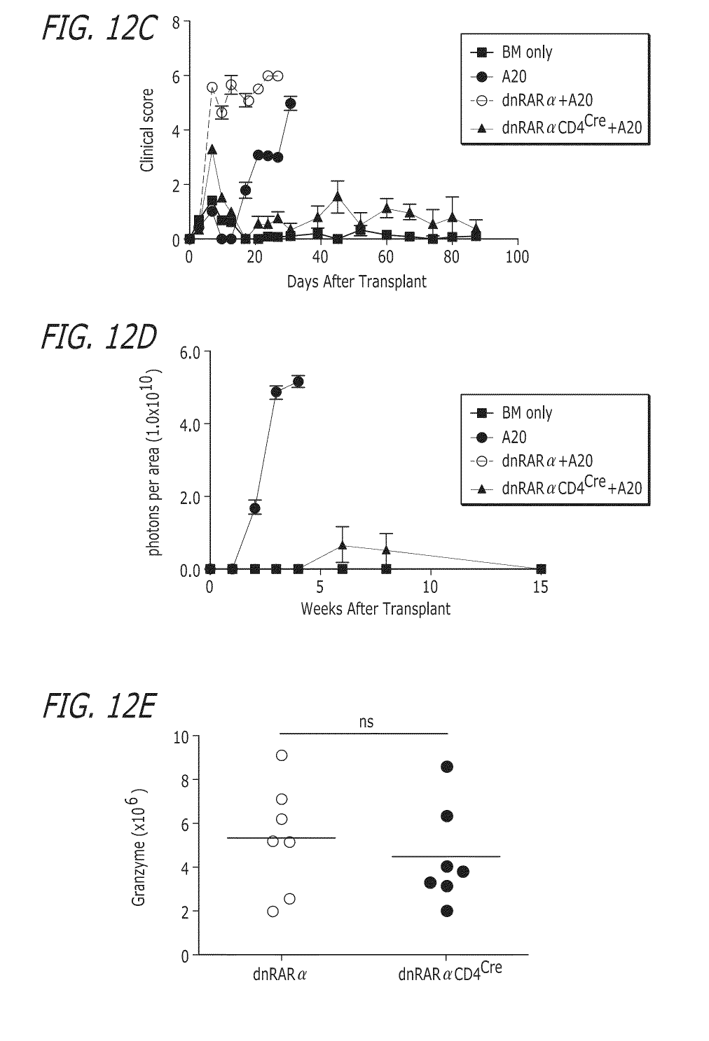

To determine whether dnRARα-CD4^{Cre} donor T cell expression would adversely affect graft-versus-lymphoma (GVL) activity, lethally irradiated BALB/c recipients were given B6 TCD-BM and A20^{luc} lymphoma cells on day 0. Groups were given splenocytes from dnRARα or dnRARα-CD4^{Cre} mice. All mice given A20^{luc} and TCD-BM alone died within 38 days due to tumor burden (FIG. 12A-D). Mice given dnRARα T cells all died of GVHD by day 31 with rapid weight loss (FIG. 12B) and high clinical scores (FIG. 12C) but with showed no or minimal initial tumor growth (FIG. 12D). Most of mice given A20^{luc} and dnRARαCD4^{Cre} splenocytes showed minimal initial tumor growth followed by regression with minimal evidence of GVHD mean weight curves or clinical scores.

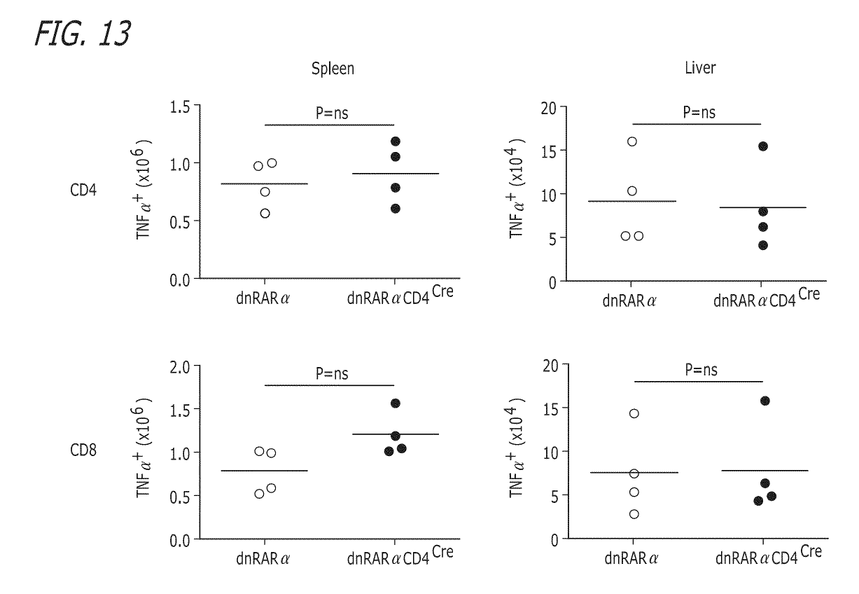

To elucidate the potential mechanisms of retained GVL effects in recipients given A20^{luc} and dnRARα-CD4^{Cre} splenocytes, we tested the granzyme B and perforin cytotoxic pathways on donor T cells. Granzyme B expression in day 14 splenic CD8⁺ T cells was largely preserved in dnRARα-CD4^{Cre} splenocytes (FIG. 12E). No differences were seen in perforin expression of CD8⁺ T cells (not shown). No significant differences in the number of TNF-α-secreting cells, associated with GVL effects, were seen in day 6 splenic or hepatic dnRARα-CD4^{Cre} vs dnRARα CD4⁺ and CD8⁺ T cells (FIG. 13).

Taken together, the present specification demonstrated that GVHD induces RA production by donor-derived DCs and macrophages in the intestines. RA signaling in donor T cells was up-regulated, contributing to donor T cell accumulation in GVHD target organs, accelerating the disease. Conversely, inhibition of RA signaling in donor T cells demonstrated reduced GVHD capacity, associated with reduced intestinal-homing receptor expression, decreased Th1 Teff proinflammatory cytokine production, and higher Tregs numbers. Despite these beneficial effects, A20 GVL effects were preserved.

RALDH, expressed in gut-associated DCs and intestinal epithelial cells, is a pivotal enzyme for RA synthesis. We determined RALDH1 and RALDH2 up-regulation in GVHD mice in the small intestine on day 14 post-BMT. ALDEFLUOR™ staining confirmed the up-regulation of ALDH enzymatic function in day 14 post-BMT small intestine and MLN macrophage and DCs. RA concentrations and signaling in donor T cells were significantly increased, suggesting that blocking either RA production or donor T cell signaling might be a useful approach to reduce GVHD. With respect to the latter, we report the novel observation that selective diminution of RA signaling in donor T cells prevents GVHD lethality, whereas RA supplementation had the opposite effect. Pathological analysis revealed the importance of RA signaling in the development of intestinal GVHD. GVHD protective effects in other organs may be due to reduced gut inflammation and permeability, which affects systemic GVHD injury and lethality. Vitamin A deficiency reduces intestinal but exacerbates liver GVHD and does not protect from overall GVHD lethality. Notably, vitamin A deficient mice have a higher level of endogenous inflammation than non-deficient mice, which we overcame by using a selective approach to preventing RA signaling in donor T cells.

We determined that selective diminution of RA signaling in donor T cells reduces early post-BMT expression of donor T cell CXCR3, α4β7 and CCR9, crucial factors for GVHD-induced gut injury. Additionally, dnRARα-CD4^{Cre} T cells had a skewed Th2 profile with lower levels of Th1 and Th17. Paracrine production of cytokines from donor T cells had no impact on Th1-Th2-cell differentiation of dnRARα-CD4^{Cre} T cells, indicating that reduction in Th1 differentiation was T cell intrinsic. RA inhibits Th1 generation and facilitates Th2 generation directly or indirectly though in our studies, Th2 skewing in recipients of dnRARα-CD4^{Cre} T cells was not observed. Th2 polarization regulates acute GVHD severity although acute GVHD may not be ameliorated by a reduction in Th1 and IFN-γ only. Nonetheless, diminished Th1 differentiation of donor T cells from dnRARαCD4^{Cre} vs dnRARα donor T cells may have contributed to reduced GVHD lethality. Because RA effects seen in dnRARα-CD4^{Cre} also may influence donor T cell signaling via RARα, RARβ and RARγ due to effects on sequestration of RXRs by dnRARα.

RA has been shown to promote TGFβ-mediated Foxp3 conversion of naïve T cells and stabilize Foxp3 expression in natural Tregs and inducible Tregs. Somewhat surprisingly, dnRARα-CD4^{Cre} donor T cells enhanced the generation of Tregs both from donor BM and T cells. Neither donor T cell autocrine nor paracrine cytokine production inhibited Treg generation/expansion in recipients of dnRARα-CD4^{Cre} T cells post-BMT. Therefore, high inflammatory cytokines associated with GVHD per se is not conducive to in vivo Treg generation and that approaches that reduce GVHD will favor inductive Treg generation and/or expansion, offsetting any deleterious effect of diminished RA signaling in donor T cells. The overall higher Treg frequencies in mice given dnRARα-CD4^{Cre} T cells may account for the greater reduction in pathologic scores of the gastrointestinal tract seen in the context of dnRARα-CD4^{Cre} donor T cells, even though RA is known to promote α4β7 expression on inducible Tregs. Alternatively or in addition, Teffs from dnRARα-CD4^{Cre} mice may be eliminated or fail to accumulate in GVHD target organs, as has been shown for CD8⁺ T cell accumulation at late but not early times after antigenic stimulation. Together, reduced donor Th1 cytokine differentiation and T cell expression of intestinal homing receptors coupled with higher Treg numbers (and hence a favorable Treg:Teff ratio) may each contribute to the lowered GVHD mortality rate seen with dnRARα-CD4^{Cre} vs dnRARα T cell administration.

Importantly, the GVL effect against A20 lymphoma was maintained despite markedly decreased GVHD lethality in BALB/c recipients of B6 dnRARα-CD4^{Cre} splenocytes versus wild type splenocytes. This GVL effect does not appear to be susceptible to Treg mediated suppression. The production of perforin-granzyme from CTL cells and TNFα from dnRARα-CD4^{Cre} donor T cells are preserved. Both perforin-granzyme and TNFα mediate the GVL effect. In spite of our recent findings that diminished RA signaling in CD8⁺ T cells dampened late CD8⁺ T cell expansion and clonal accumulation in a tumor microenvironment, GVL activity appeared intact. These differences in anti-tumor responses of dnRARα-CD4^{Cre} donor T cells may be due to the Teff mechanisms required (e.g. CD4⁺ T cells can recognize MHC class II⁺ A20 cells and high sensitivity to perforin-granzyme B mediated cytolysis) and the high frequency of alloresponsive (and hence A20 responsive) vs syngeneic anti-tumor responsive T cells.

In conclusion, these studies demonstrate that the enhanced RA synthesis and RA signaling in donor T cells during GVHD augments disease severity. Because inhibiting RA signaling in donor T cells attenuates GVHD lethality while preserving A20 GVL activity, selectively targeting of the RA signaling pathway or the ALDH enzymes responsible for RA production may be useful for GVHD prevention or therapy.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A method of treating a transplant rejection, the method comprising administering to an individual in need thereof a therapeutically effective amount of a RAR antagonist, thereby treating the transplant rejection in the individual.

2. The method according to claim 1, wherein the RAR antagonist is a RARα antagonist and/or a RARγ antagonist.

3. The method according to claim 2, wherein the RARα antagonist is

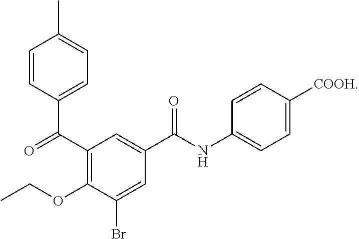 [SMILES: CCOc1c(Br)cc(C(=O)Nc2ccc(C(=O)O)cc2)cc1C(=O)c1ccc(C)cc1]

4. The method or use according to claim 2, wherein the RARγ antagonist is

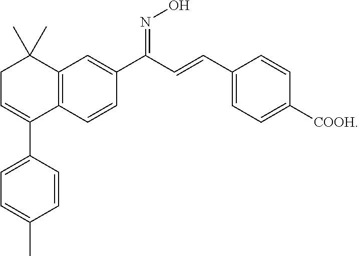 [SMILES: Cc1ccc(C2=CCC(C)(C)c3cc(C(/C=C/c4ccc(C(=O)O)cc4)=N/O)ccc32)cc1]

5. The method according to claim 1, wherein the transplant rejection is a graft-versus-host-disease (GVHD) disorder.

6. The method according to claim 5, wherein the GVHD disorder is an acute GVHD disorder or a chronic GVHD disorder.

7. The method according to claim 1, wherein the transplant rejection is a hyperacute rejection, an acute rejection, or a chronic rejection.

8. The method according to claim 1, wherein the therapeutically effective amount is about 0.01 mg/kg/day to about 100 mg/kg/day.

9. The method according to claim 1, wherein the therapeutically effective amount is about 0.1 mg/m²/day to about 100 mg/m²/day.

10. The method according to claim 1, wherein administration causes reduction of at least one symptom of transplant rejection selected from inflammation, fatigue, dizziness, malaise, elevated fever and high body temperature, extreme sensitivity to cold in the hands and feet, weakness and stiffness in muscles and joints, weight changes, digestive or gastrointestinal problems, low or high blood pressure, irritability, anxiety, or depression, infertility or reduced sex drive (low libido), blood sugar changes, and the destruction of an organ or tissue.

11. The method or use according to claim 10, wherein the administration causes a reduction in at least one symptom of inflammation selected from edema, hyperemia, erythema, bruising, tenderness, stiffness, swollenness, fever, a chill, congestion of the respiratory tract including nose and bronchi, congestion of a sinus, a breathing problem, fluid retention, a blood clot, a loss of appetite, an increased heart rate, a formation of granulomas, fibrinous, pus, or non-viscous serous fluid, a formation of an ulcer, or pain.

12. A method of treating graft-versus-host disease, the method comprising administering to an individual in need thereof a therapeutically effective amount of a RAR antagonist, thereby treating the graft-versus-host disease in the individual.

13. The method according to claim 12, wherein the RAR antagonist is a RARα antagonist and/or a RARγ antagonist.

14. The method according to claim 13, wherein the RARα antagonist is

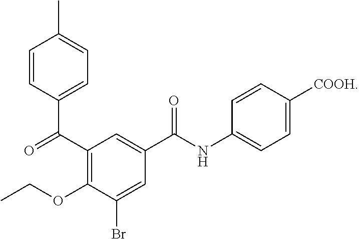 [SMILES: CCOc1c(Br)cc(C(=O)Nc2ccc(C(=O)O)cc2)cc1C(=O)c1ccc(C)cc1]

15. The method or use according to claim 13, wherein the RARγ antagonist is

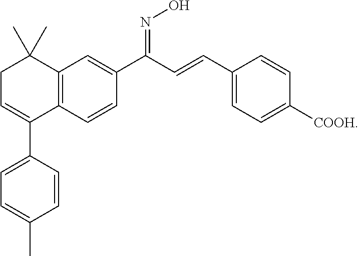 [SMILES: Cc1ccc(C2=CCC(C)(C)c3cc(C(/C=C/c4ccc(C(=O)O)cc4)=N/O)ccc32)cc1]

16. The method according to claim 12, wherein the GVHD disorder is an acute GVHD disorder or a chronic GVHD disorder.

17. The method according to claim 12, wherein the therapeutically effective amount is about 0.01 mg/kg/day to about 100 mg/kg/day.

18. The method according to claim 12, wherein the therapeutically effective amount is about 0.1 mg/m²/day to about 100 mg/m²/day.

19. The method according to claim 12, wherein administration causes reduction of at least one symptom of transplant rejection selected from inflammation, fatigue, dizziness, malaise, elevated fever and high body temperature, extreme sensitivity to cold in the hands and feet, weakness and stiffness in muscles and joints, weight changes, digestive or gastrointestinal problems, low or high blood pressure, irritability, anxiety, or depression, infertility or reduced sex drive (low libido), blood sugar changes, and the destruction of an organ or tissue.

20. The method or use according to claim 19, wherein the administration causes a reduction in at least one symptom of inflammation selected from edema, hyperemia, erythema, bruising, tenderness, stiffness, swollenness, fever, a chill, congestion of the respiratory tract including nose and bronchi, congestion of a sinus, a breathing problem, fluid retention, a blood clot, a loss of appetite, an increased heart rate, a formation of granulomas, fibrinous, pus, or non-viscous serous fluid, a formation of an ulcer, or pain.
